(19) United States
(12) Patent Application Publication
(10) Pub. No.: US 2009/0234459 A1
(43) Pub. Date: Sep. 17, 2009

(54) MEDICAL DEVICE FOR INSERTION INTO A JOINT

(75) Inventors: Sune Lund Sporring, Naestved (DK); Ole Olsen, Charlottenlund (DK); Jes Bruun Lauritzen, Kobenhavn K (DK); Tom Logstrup Andersen, Roskilde (DK); Povl Brondsted, Koge (DK); Klaus Bechgaard, Kobenhavn O (DK); Jan Guldberg Hansen, Greve (DK); Henrik Jensen, Vanlose (DK); Kristoffer Almdal, Roskilde (DE); Soren Mouritsen, Birkerod (DK)

Correspondence Address: NIXON & VANDERHYE, PC, 901 NORTH GLEBE ROAD, 11TH FLOOR, ARLINGTON, VA, 22203, US

(73) Assignee: Cartificial A/S, Vedbaek (DK)

(21) Appl. No.: 12/331,335

(22) Filed: Dec. 9, 2008

Related U.S. Application Data
(63) Continuation of application No. 11/917,502, now abandoned.

(30) Foreign Application Priority Data
Jun. 14, 2005 (DK) PA 2005 00870
Sep. 2, 2005 (DK) PA 2005 01228

(51) Int. Cl. A61F 2/30

(52) U.S. Cl. 623/18.11

(57) ABSTRACT

The present invention relates to implants used for alleviating and/or preventing conditions relating to damaged joints involving articulating surfaces. The implants comprise fibre of polymer and/or metal, and can be used as an artificial joint, as part of an artificial joint or as an artificial joint spacer made to replace the missing cartilage or to improve the slidability between two natural and/or artificial components of the body, or between a natural and artificial component. The product of the invention can be used to partly or entirely coat medical products or to make up implant partly or entirely.

### CROSS REFERENCE TO RELATED APPLICATIONS

This application is a continuation of application Ser. No. 11/917,502 that was submitted to the United States Patent and Trademark Office on Dec. 13, 2007, which is a U.S. National Stage entry under 35 U.S.C. § 371 of and claims priority to International Patent Application No. PCT/DK2006/000343, filed Jun. 14, 2006, designating the United States of America and published in English on Dec. 21, 2006 as WO 2006/133711, which claims the benefit of Denmark Application No.: PA 2005 00870, filed Jun. 14, 2005, and Denmark Application No.: PA 2005 01228, filed Sep. 2, 2005, each of which is hereby incorporated by reference in its entirety.

### FIELD OF INVENTION

The present invention relates to a method and a medical device for alleviating and/or preventing conditions relating to damaged joints involving articulating surfaces. Especially the present invention relates to medical products which are coated partly or entirely by one or more polymeric components and to medical products which comprises metal and polymeric materials. The product of the present invention can be used as an artificial joint, as part of an artificial joint or as an artificial joint spacer made to replace the missing cartilage or to improve the slidability between two natural and/or artificial components of the body, or between a natural and artificial component of the body. All patent and non-patent references cited in the application, are also hereby incorporated by reference in their entirety.

### BACKGROUND OF INVENTION

At present, joint damage, such as cartilage damage, is treated by replacing the joint with an artificial joint. However, serious complications are caused by the replacement of artificial joints, in particular a high occurrence rate of loosening problems resulting in breakage of the bones around the artificial joint. In the case of cartilage damage a repair with cartilage substitution placed into intact bones is to be preferred instead of replacing the entire joint.

In particular, the invasive character of the fixation of the prostheses such as anchoring of the prosthesis with screws and pins results in numerous side-effects such as risk of infection, loosening as mentioned above, damage on excising bone due to interruption of blood supply and necrosis.

A device for replacement within a joint should preferably enable the normal function and movements of the joint. Weight-bearing joints, in which movement in more than one direction takes place, are normally rather difficult to replace.

A prosthetic device should enable the normal movement of the joint. During walking, the normal movement of for example the hip joint corresponds to about 37°-41° flexion/extension, 2°-14° adduction/abduction and a rotation of about 2-16°. During movement from standing to sitting position a flexion of hip joint corresponds to a movement from 0 to 90 degrees. When studying the movement of femoral caput to the acetabulum the latter movement includes a rotation of 90 degrees.

Many medical devices are implanted into load-bearing joints such as knees, hips, etc, or utilised in the human body where mechanical function provide high strength or shape stability such as heart valves, breast prosthesis, stent, catheter, etc. As such, these medical devices must be very strong and possess a high degree of wear resistance. Prosthetic medical devices manufacturers constantly work toward developing better products by improving their physical properties. Improved wear resistance, for example, is a desirable quality to impart to a prosthetic medical device. Improving wear resistance without losing strength or causing oxidative degradation is a difficult balance to obtain.

A need for improved prosthetic medical devices with improved wear resistance exists.

Many implants produce small particles on the surface of the implant when subjected to motion. The particles are liberated from the implant as abrasion caused by the motion of the implant and friction towards other components within the body. These particles can give rise to inflammation within the individual where the implant is implanted. In a test controlling the wear characteristic of a hip-joint implant with traditionally polyethylene (PE) acetabular cups, about 10¹² particles are produced pr. million of movements (cycles).

The use of ceramic or cross-linked PE cups slightly reduces the number of particles produced. Still the produced particles give rise to inflammation and also there is a risk that the implant becomes loose.

An implants produced with a smooth UHMWPE coating according to the present invention will only produce 5*10⁸ or less particles pr. million cycles. Thus a lesser degree of inflammation and a longer life of these implants are some of the results when implanted into an individual.

### SUMMARY OF INVENTION

The present invention relates to medical devices which may be used in damaged joints involving articulating surfaces.

The medical device may comprise at least one fabric of one or more polymer fibre and/or of one or more metal fibre and/or a 3D network of polymer fibre and/or metal fibre. The at least one fabric may also be at least one 2D network. Fabrics, 2D networks and 3D networks may be used together in one medical device.

The volume within the fibres making up the device may be filled up with one or more polymer optionally further comprising one or more metal components or the volume is filled with a metal or metal alloy. The volume to be filled up with one or more polymer optionally further comprising one or more metal components can be determined according to the shape and total volume of the final device ready to use. The device may be subjected to size-reducing processes during preparation, thus the initially volume defined by the fibres of the fabrics and/or networks can be larger than the corresponding volume within the final device.

Biocompatible polymers and biocompatible metals can be used to make up the device. Polymers and metals are described elsewhere herein.

A device or part of a device made up according to the present invention may be used for any implant. Especially implants to be positioned into joints can be made, such as an acetabular cup, a spacer to be located between an acetabular cup and a head of hip stein, or an interpositional arthroplasty. The head of the hip stein can be made of metal or a ceramic material. The device may also be a cup shaped spacer which is positioned between the natural femoral stem or a metal femoral stem and a natural acetabular cup or a prosthetic acetabular cup.

The medical may comprise at least a first surface area, wherein at least a first polymeric component optionally further comprising one or more metal components is attached to or coated on the first surface area. The material to coat a medical implant can be made with fabrics, 2D networks and/or 3D networks of polymer fibre and/or metal fibre where the fibre are connected to each other or with another polymer and/or metal material as described herein.

A medical device produced from the materials described herein as well as devices produced by other methods may be coated or covered partly or entirely with a polymeric material of the present invention, the polymeric material optionally comprises a metal component.

The medical device may have an upper surface, a lower surface and at least one edge and wherein at least the one edge may be sealed by a collar. The collar can be made of a material according to the present invention, e.g. it can be made with fabrics, 2D networks and/or 3D networks of polymer fibre and/or metal fibre where the fibre are connected to each other or with another polymer and/or metal material as described herein.

Also the collar may be made of a first second and/or a third polymeric component optionally further comprising one or more metal components.

A surface of the device can be made smooth by using the material according to the present invention. The device comprises at least a first and a second side, wherein the at least first side is made of a first polymeric component optionally further comprising one or more metal components and wherein the part of or the entire of the first and/or second side has a frictional resistance of less than 0.5 Newton.

The smooth surface can be made by using a material according to the present invention, e.g. it can be made with fabrics, 2D networks and/or 3D networks of polymer fibre and/or metal fibre where the fibre are connected to each other or with another polymer and/or metal material as described herein.

The smooth surface may further include one or more cavities.

The device may have at least one through-going perforation which is not for ligaments. The perforation may have a diameter of at least 0.01 mm and can conduct liquid from one first side of the device to another second side of the device. The liquid may be synovial fluid or a physiological salt solution and/or another biocompatible liquid.

The device may also be equipped with an inner volume between the first and second side and where said liquid can be within this volume. This inner volume may further comprise a network of at least one polymeric and/or metal component, e.g. polymeric fibres and/or metal fibres.

The surface of a medical device according to the present invention may be a self-healing surface. The medical device comprises a first polymeric component optionally further comprising one or more metal components which may be self-healing when subjected to injury before implantation, during implantation and/or after implantation.

The self-healing surface can be made by using a material according to the present invention, e.g. it can be made with fabrics, 2D networks and/or 3D networks of polymer fibre and/or metal fibre where the fibre are connected to each other or with another polymer and/or metal material as described herein. Other polymers described herein can also be used to produce a self-healing surface.

The surface material may be any material described herein, e.g. a composite polymeric component optionally her comprising one or more metal components. The device can include a microencapsulated healing agent that is released upon injuries hereby polymerization of the polymeric component is triggered by contact with an embedded catalyst/initiator. The healing agent can be monomers of the first polymeric component and/or of a second polymeric component.

The medical device according to the present invention may be two parts or units which fit into each other, the device comprise at least a first unit with at least a convex surface and a second unit with at least a concave surface, where the convex and concave surface is congruent with each other and the first unit fit partially or entirely into the second unit, and the first and/or second unit comprises at least one polymeric component optionally further comprising one or more metal components.

Each unit can be made by using a material according to the present invention, e.g. it can be made with fabrics, 2D networks and/or 3D networks of polymer fibre and/or metal fibre where the fibre are connected to each other or with another polymer and/or metal material as described herein.

The first and second unit can have substantially similar sizes, or be of different sizes. The units may be movable when compared to each other, and the units can continue to be in contact during a movement of the body and/or return to be in contact when a movement is finished.

The medical device according to the present invention may comprise at least an upper layer, a first middle layer and a lower layer, wherein the upper layer and said lower layer is made from one uninterrupted piece of at least a first polymeric component optionally further comprising one or more metal components.

The material to make up the device may be a fabric, 2D network and/or 3D network which can be constructed in one piece, which is folded around an axis and hereby forming the upper and lower layer.

The upper and lower layer of the device may be similar or substantially similar in size, and may be formed from a tube folded around the axis. The tube may have a uniform diameter along the length, or may contract in a first and/or a second end of said tube, e.g. the tube contract in the axis. The device may in the axis further comprise at least one aperture.

The medical device according to the present invention may also be thicker at the periphery of the device. The device may comprise at least a first polymeric component optionally further comprising one or more metal components, wherein the device has a middle area which at least in one dimension is surrounded by an outer area, and wherein the thickness of the outer area at least partly is larger than the thickness of the middle area, and the outer area ends in at least one edge of said device.

The device may have any suitable shapes, e.g. cup-shaped or approximately cup-shaped, and wherein a middle area is the top and an outer area is a skirt. The skirt may be thicker that the top.

The device may also be a layered device. The device in a layered structure may comprise
at least one upper layer of said first polymeric component optionally her comprising one or more metal components,
a middle layer of said second polymeric component optionally further comprising one or more metal components, and
at least one lower layer of said third polymeric component optionally further comprising one or more metal components, where the chain length of the first polymeric component and the third polymeric component is longer than the chain length of the second polymeric component.

In a layered device as described above, each layer may itself be composed of at least two sub-layers of polymeric fabric and/or a 2D network and/or a 3D network, each sub-layer optionally further comprising one or more metal components constructed of the polymeric components described herein, and at least one layer of polymeric film optionally further comprising one or more metal components, said polymeric film constitutes a layer between the two layers of the polymeric fabrics and/or 2D network and/or 3D network.

Also the middle layer, the film may further comprise one or more metal components.

The metal used in the device may be one or more metals selected from the group of metal and metal alloys of titanium, gold, silver, chromium-cobaltum, zirconia, cobalt-chromium-molybdenum alloy and Stainless Steel alloys and/or a ceramic of one or more of these metals and alloys. Other metals may also be included e.g. in the alloys.

The polymers and metals including alloys used may be in the form of e.g. a powder, granulate, chopped fibres, long fibres, 2D structural components like plates, 3D structural components like shaped plates or hemicircles with holes. Also a combination of these forms may be used.

Instead of using fabrics, 2D networks and/or 3D networks of polymer fibre and/or metal fibre to produce the devices as described above, it may also be possible to use polymers and/or metals which are suitable for injection moulding, these materials may further include components of polymer and/or metal, as described herein.

### DESCRIPTION OF DRAWINGS

FIG. 1 illustrates a longitudinal section of a cup-shaped medical device.

FIG. 2 illustrates a cross-section of a cup-shaped medical device.

FIG. 3 illustrates a longitudinal section of a cup-shaped medical device. The device has a shirt in which the material is thicker than the material at the top of the device.

FIG. 4 illustrates a cup-shaped medical device. The line denoted “E” illustrates equator, which is the border between the top “T” and the shirt “S”.

FIG. 5 illustrates a cup-shaped medical device (2) with a collar (1).

FIG. 6 illustrates a longitudinal section of a cup-shaped medical device. The device has a shirt in which the material is thicker than the material at the top of the device. Furthermore the device has a collar with a marker.

FIG. 7 illustrates the edge of the medical device in FIG. 6. The edge is enclosed by a collar, and this collar supports a marker (3).

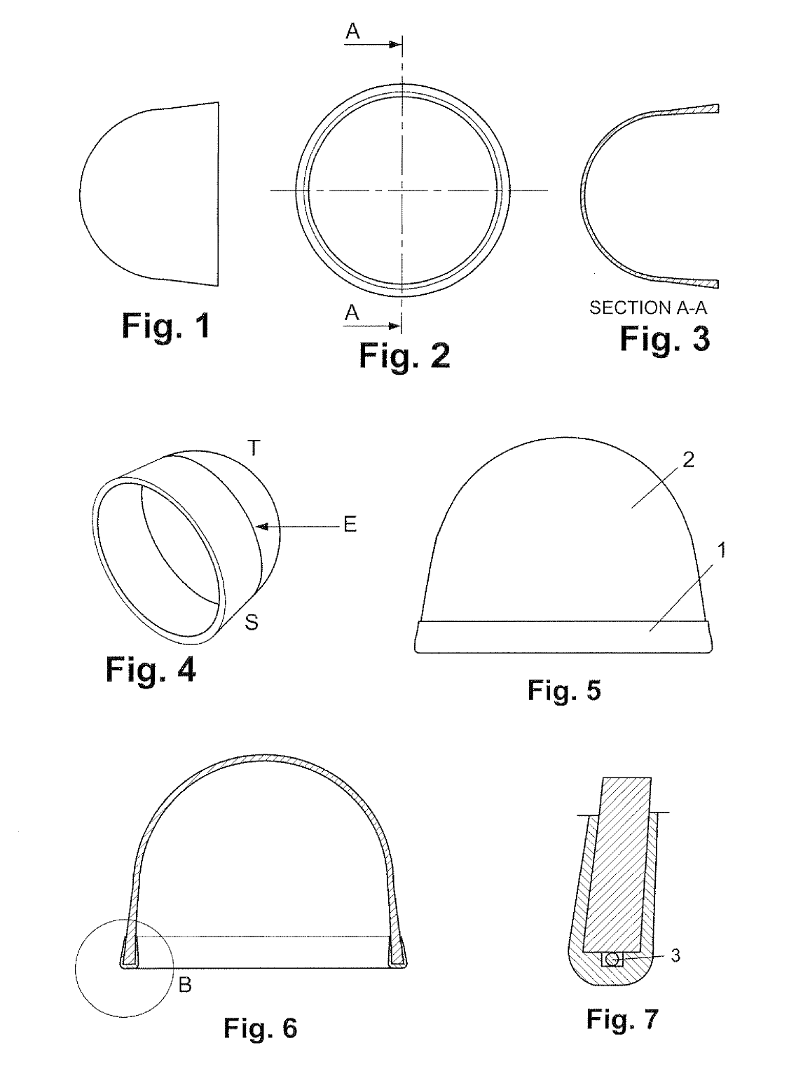

### DETAILED DESCRIPTION OF DIE INVENTION

The present invention provides a desirable balance of improved wear resistance and high tensile strength and toughness in the polymeric compositions used for medical devices. The implants may also include metal e.g. as a composite with polymeric materials. It has been discovered that wear resistance can be improved without sacrificing other desirable properties such as toughness or strength by controlling the amount of different polymeric and/or metal substrate comprising the prosthetic device. The products of the invention has a high tensile strength and improved wear resistance as well as the capability to absorb shocks, impacts and pressure load, also it reduces the amount of tearing off.

The medical device may be designed to occupy the pelvis cup; to occupy at least part of the intra-articular cavity to partly or completely fill the role of natural cartilage within a joint; and/or to be an interpositional arthroplasty. The devices or their units may be designed so as to occupy the whole of the cavity or merely a portion of the intra-articular cavity, such as the portion of the cavity where cartilage is worn or where much of the pressure is exerted.

The medical devices may also be designed to replace at least part of a bone within a joint together with the intra-articular cavity to partly or completely fill the role of the bone which is replaced and to partly or completely fill the role of natural cartilage within a joint.

The material for the medical devices is primary polymers although other materials as described elsewhere herein can be used. The other materials e.g. metals may be used together with the polymers.

In an aspect of the invention a medical device comprises at least one fabric of one or more polymer fibre and/or of one or more metal fibre and/or a 3D network of polymer fibre and/or metal fibre. The polymer fibre and metal fibre which may be used are described elsewhere herein.

In an embodiment the device comprises an area at least defined by the outermost fibres of the network, this area is filled up with one or more polymer optionally further comprising one or more metal components or said area is filled with a metal or metal alloy. The polymer and metal components used may be anyone described elsewhere herein. The fibres of the network may during the production process of the device be located substantially in the position where the fibres will be located in the final device.

Also the fibres making up the network may during the production process be located in different positions when compared to the location of these fibres in the final device. Hereby the volume to be filled up may be defined by the outermost fibres of the network and is a volume defined by the corresponding volume of the network In the final device, hereby the volume defined by the network of fibres is larger In the production process than the volume defined by the final device.

In the production process heat and/or pressure may be used to connect polymers and/or metals in different forms to each other.

In an embodiment the polymer fibre and/or the polymer optionally further comprising one or more metal components may be a polyolefin, such as polyethylene, such as UHMWPE. Also other polymers as described elsewhere herein may be used. The polymer fibre and the polymer optionally further comprising one or more metal components can be different types of polymers. Also the metal fibre and the metal components can be different types of metal.

In an aspect of the invention a medical device comprises at least a first surface area, wherein at least a first polymeric component is attached to the first surface area.

In an embodiment of the device, the first surface area is a part or the entire surface area of a first volume, where the first volume is smaller than the final volume and where the final volume is the total volume of the device before implantation, and wherein the difference between the final volume and the first volume is a volume made up by at least a first polymeric component a first metal component or a combination of a first polymeric component and a first metal component.

At least a first polymeric component and/or a first polymeric component together with a first metal component can coat a part or the entire surface of an implant. A second and/or third polymeric component or a combination of a second and/or third polymeric component together with a second and/or third metal component may coat the same surface part as coated by the first polymeric component, optionally together with a first metal component, or the second and/or third polymeric component, optionally together with a second and/or third metal component, may coat a part of the surface or different parts of the surface than coated by the first polymeric component optionally with a first metal component.

The term “first volume” is used to describe the produced implant or part of an implant at a stage in the production where only a coating according to this invention is lacking on the implant. This definition does not exclude the possibility of treating the coated implant e.g. by irradiation or by a liquid solution and thus the final volume being the coated implant slightly can change in volume.

In an embodiment the first volume has a shape which corresponds to the final volume of the device before implantation. In this case “corresponds to” can mean having exactly the same shape just with a smaller volume, or can mean that the overall shape Is similar when comparing the first volume and the final volume, but in one or more zones the two volumes correspond to a lesser degree to each other than in other zones.

In a further embodiment the first volume corresponds to at least 50% of the final volume, such as at least 60%, such as at least 70%, such as at least 80%, such as at least 85%, such as at least 90%, such as at least 95%, such as at least 97%, such as at least 99%. This calculation can be made based on the overall shape of the first and final volume or can be based on a comparison between different although related zones of the first and final volume of the implant.

In an embodiment the first volume corresponds to a lesser degree to the final volume in areas where the at least first polymeric component optionally together with a first metal component, is attached. If only a part of the surface of the implant is to be coated with the first polymeric component and/or second polymeric component and/or third polymeric component optionally together with a first, second and/or third metal component respectively, the zones including the surface area to be coated will correspond to a lesser degree to the final volume of the implant than zones located elsewhere in the implant.

In an embodiment the first volume ter comprises different first zones, and where the different first zones are smaller volumes of the first volume and the first volume in different first zones corresponds to a different degree to related final zones of the final volume, and where the final zones are smaller volumes of the final volume. The zones of the first volume and final volume are not of a specific volume, but are to be determined from implant to implant. When a zone of a first volume corresponds to a zone of a final volume, this means the two zones are located in similar areas of the implant when taking into account that the final volume is larger than the first volume.

In an embodiment the volume of the first zones corresponds to at least 50% of the volume of the related final zones, such as at least 60%, such as at least 70%, such as at least 80%, such as at least 85%, such as at least 90%, such as at least 95%, such as at least 97%, such as at least 99%.

In another embodiment the volume of the first zones corresponds to a different degree to the volume of the related final zones and where the different degree is selected between 50-100%, such as 60-100%, such as 70-100%, such as 80-100%, such as 90-100%, such as 95-100%, such as 50-90%, such as 60-90%, such as 70-90%, such as 80-90%, such as 50-80%, such as 60-80%, such as 70-80%, such as 50-70%, such as 60-70%. Some zones of the first volume may be relatively smaller than other zones when compared to the related zones of the final volume.

In another embodiment the first volume further correspond to the final shape of the final volume. Only slight differences exist between the shape of the volume and the shape of the final volume.

In an embodiment the first surface area comprises the outer surface of one or more of the first zones. The number of zones constituting the first surface area can be calculated if a zone is defined as a volume of the implant where the zone(s) next to this zone have or is going to have a coating that differs in thickness or overall polymeric composition when compared to the zone of interest.

In an embodiment the at least first polymeric component is attached to the entire outer surface of the first volume. An implant can be coated over the entire outer surface with the first polymeric component, optionally together with a first metal component and/or a second polymeric component optionally together with a second my component and/or a third polymeric component, optionally together with a third metal component.

In an embodiment the device at least in the first area is subjected to wear when the device is located in an individual. An implant can be coated either in only the area(s) which are subjected to wear or also in other areas. The polymeric components, optionally together with metal components, used as coating are described elsewhere herein and can be one or more polymeric components optionally together with one or more metal components.

In an embodiment the first volume of Me device may be made partly or entirely of polymer, bone and/or metal. The implant to be coated can be made of any material which can be coated by one or more polymeric components, optionally together with metal components, as described elsewhere herein.

In implants composed of different units, one or more of these units can be coated partly or entirely by the polymeric components described herein, the polymeric components may comprises metal components as also described elsewhere herein.

In an embodiment the first polymeric component, optionally together with one or more metal components, constitute at least 1% of the entire outer surface of the device, such as at least 3%, such as at least 5%, such as at least 8%, such as at least 10%, such as at least 15%, such as at least 20%, such as at least 25%, such as at least 30%, such as at least 35%, such as at least 40%, such as at least 50%, such as at least 60%, such as at least 70%, such as at least 80%, such as at least 90%, such as at least 99%. A is calculation is based on a comparison of the outer area of the first volume and the outer area of the final volume.

In another embodiment the first polymeric component constitute at least 1% of the diameter of the final volume of the device, such as at least 3%, such as at least 5%, such as at least 8%, such as at least 10%, such as at least 15%, such as at least 20%, such as at least 25%, such as at least 30%, such as at least 35%, such as at least 40%, such as at least 50%, such as at let 60%. This calculation is based on a comparison of the diameter of the first volume and the diameter of the final volume, and can be an overall calculation or a calculation within one or more zones.

In an embodiment the first polymeric component, optionally together with metal components, may be located at the outside of the device in areas that is subjected to wear when located in a body. The implant can be coated only in areas that is subjected to wear, or in other areas too. It is important to coat the implant in the areas subjected to wear.

In an embodiment the first polymeric component, optionally together with metal components, is in the form of a fabric. The first, second and third component, optionally together with metal components, can be in any form described herein and can be combined in any way as described elsewhere herein.

In an embodiment the first and the second polymeric component comprises the same monomeric component or the same components of the composite material, and the first and second polymeric components are differently crystallized within the implant ready to implant. The first and the second polymeric component may comprise a first and/or second metal component. These metal components may be similar or different types of metal or the same type of metal although in pieces of different sizes e.g. short fibre and long fibre.

Also the third polymeric component can be of the same monomeric component or the same components of the composite material as the first and/or second polymeric component, and/or be differently crystallized within the implant ready to implant when compared to the first and/or second polymeric components. The third polymeric component may comprise a third metal component. This metal component may be of similar or different types of metal than the first and/or second metal component, or the same type of metal although in pieces of different sizes e.g. shorter fibre and/or longer fibre

In an embodiment the first and the second polymeric component comprises the same monomeric component or the same components of the composite material and the first and second polymeric components have different morphology within the implant ready to implant.

Also the third polymeric component can be of the same monomeric component or the same components of the composite material as the first and/or second polymeric component and have a differently morphology within the implant ready to implant when compared to the first and/or second polymeric components.

In an embodiment the first polymeric component and/or the second polymeric component and/or the third polymeric component each optionally comprising one or more metal components may be attached to the first volume by ultrasound welding, laser welding, heating and/or gluing. Any method to secure the polymeric components to the first volume can be used.

In an embodiment the first polymeric component and/or the second polymeric component and/or the third polymeric component each optionally comprising one or more metal components are attached to each other before attached to the first volume. The polymeric components, optionally comprising one or more metal components, can be attached to each other e.g. in a layered structure as described elsewhere herein, but also other compositions described herein can be used.

In an embodiment the first polymeric component and/or the second polymeric component and/or the third polymeric component each optionally comprising one or more metal components, are attached to the first volume one polymeric component at a time. The polymeric components, optionally comprising one or more metal components, can be attached to the first volume of the implant in any order suitable for the implant thus the first polymeric component need not be the polymeric component situated closest to the first volume. The number and order of the polymeric components, optionally comprising one or more metal components, are not limited.

In an embodiment the number of layers of the polymeric components, optionally comprising one or more metal components, includes more than one layer of one of the polymeric components, optionally comprising one or more metal components, and each of the layers are attached to the first volume one polymeric component at a time. Preferred number of layers is described elsewhere herein.

In a embodiment the said device comprises a part of or an entire hip joint prosthesis with a natural or metal and/or ceramic femoral stem articulating against an acetabular cup to be placed in the pelvis. The acetabular cup may be a PE acetabular cup, a UHMWPE acetabular cup or a metal acetabular cup, and the metal or ceramic femoral stem and/or the acetabular cup may be covered according to the possibilities described elsewhere herein.

In an aspect of the invention a device comprises an acetabular cup or socket to be inserted in the pelvis cup, where the acetabular cup may be made of any polymeric material and which may be covered and/or reinforced with long polymeric fibre. The fibre may be in any form as described elsewhere herein.

The long polymeric fibre which may cover and/or reinforce an acetabular cup, may be in the form of one or more fabric or one and/or more layers of fibre as described elsewhere herein or may be a 3D-structure. The device comprising fabric and/or layers of fibre may be produced as described elsewhere herein. In this context a 3D-structure is a composite made of a 3D (three-dimensional) network of polymer fibre and/or metal fibre filled with and optionally entirely surrounded by a polymeric component. The 3D-network may have a knitted, crochet and/or weaved structure or may be made by any other method described elsewhere herein. The polymeric component to fill out the 3D network may be any polymer mentioned elsewhere herein.

Preferred is an acetabular cup as described above where the long polymeric fibre are UHMWPE fibre, and the polymeric material to fill out the space between the fibre has a melting point below the melting point of the used UHMWPE fibre.

More preferred is an acetabular cup as described above which may be covered with a fabric (2D-network) and/or a 3D-network of UHMWPE fibre, and where the polymeric material to fill out the space between the fibre is a polyethylene (PE) material. The PE material to fill out the space between the fibre may be only one type of PE or may be PE of different length.

In an embodiment the acetabular cup as described above may be used together with a cup-shaped spacer made in accordance with the description elsewhere herein.

In an aspect of the invention the device is part of a hip joint prosthesis or another prosthesis, and the device comprises a spacer between two parts of a prosthesis or between a part of a prosthesis and a natural bone of an individual. In a preferred embodiment a cup-shaped spacer is positioned between the natural femoral stem or a metal femoral stem of a hip joint prosthesis and a natural acetabular cup or a prosthetic acetabular cup.

In another embodiment the device is a spacer to be positioned between two natural bone parts of an individual e.g. as an interpositional arthroplasty.

In an aspect of the invention, the device includes at least a first polymeric component optionally with a first metal component and the device has a middle area which at least in one dimension is surrounded by an outer area, and wherein the thickness of the outer area at least partly is larger than the thickness of the middle area, and the outer area ends in at least one edge of the device.

In an embodiment the middle area of the device is surrounded by the outer area in two dimensions. The device may have any shape suitable to be allocated into a joint of a mammal. The device may be flat or substantially flat. Furthermore the device may cup-shaped. Other shapes are described elsewhere herein.

In another embodiment the device is homogenous in dimensions around an axis, and the axis is a central axis according to one dimension of the middle area. The axis indicates a line separating the device into two substantially equally sized units.

In an embodiment, the device is heterogeneous in dimensions around an axis, and the axis is an approximately central axis according to one dimension of the middle area. The axis indicates a line separating the middle area of the device into two substantially equally sized units. The outer area is larger on one side of the device than on other sides of the device.

In an embodiment the middle area and the at least one edge are in different planes. The device is non-homogeneous in shape and non-homogenous with respect to the planes of the edges.

In an embodiment at least one edge are in different planes. One edge is in one plane and at least another edge is in another edge, and when these planes are projected to a similar plane, this similar plane constitutes an outline of the edge or edges of the device, and the outline has a shape that is selected from a shape from triangular to circular.

The shape of the device may be any possible figure in each dimension where the shape may constitute a surface being flat, curved, waved, undulated, bent, bowed, crooked, while the overall shape of the device may be but is not limited to circular, oval, triangle, squared, rectangle, cubed, bowl, cup, crown, cap, basin, heart, egg, kidney, figure of eight, preferred shape is cup or hemispherical. The thickness of the device may also vary, as described elsewhere herein.

In an embodiment the device is cup-shaped or approximately cup-shaped, and the middle area is a top and the outer area is a skirt.

In the cup-shaped device with a rounded top, a line separating the top and the skirt is equator, and the outmost part of the skirt is the edge of the device. Equator is located around the cup-shaped device where the curvature of the cup changes to follow the tangent to the cup.

The cup-shaped or approximately cup-shaped device may have a substantially uniform thickness all over the device or the device may have various thicknesses. Preferred is a top of a first thickness and a skirt with a second thickness. The second thickness can be at least 25% larger that the first thickness, such as at least 50%, such as at least 75%, such as at least 100%, such as at least 125%, such as at least 150%, such as at least 175%, such as at least 200%, such as at least 225%, such as at least 250%, such as at least 300%, such as at least 350%, such as at least 400%, such as at least 500%.

Preferred is a cup-shaped device with a second thickness that is about 200% of the first thickness.

In an embodiment the thickness of the outer area is at least 5% larger than the thickness of middle area. The thickness of the middle area is measured at the location where the middle area is thinnest and the thickness of the outer area is measured at the location where the outer area is thickest.

The thicker outer area compared to the middle area provide the device with an increased stability in the outer area, especially when compared to a device with a uniform or substantially thickness as of the middle area. The stability of the device implies that the device to a lesser degree bent or flex in the outer area when located in the joint. Especially a cup-shaped or substantially cup-shaped device has the advantages as mentioned.

In the manufacture of the device a polymeric material optionally comprising one or more metal components as described elsewhere, has to be spread within the device. This becomes easier when the outer area is thicker than the middle area. Thus a reproducible production of uniform devices or substantially uniform devices becomes more steady, hereby increasing the quality of the devices.

A further advantage of a thicker outer area than the middle area of the device is that it is easier to finish or close the edge of the device. The device can be closed by a collar as described elsewhere herein.

A thin device increases the flexibility of the device, hereby the mammal such as a human is subjected to lesser degree of inconveniences when the device is located in a joint of this mammal. A thick outer area thus increases the stability of the device without decreasing the stability of the device.

In an embodiment the device further comprises at least a second polymeric component optionally comprising a second metal component, wherein the chain length of the first polymeric component may be longer than the chain length of the second polymeric component.

A device according to the invention comprises at least a first polymeric component and a second polymeric component, each optionally comprising one or more metal components, wherein the chain length of the first polymeric component is longer than the chain length of the second polymeric component. The first polymeric component is providing the physical properties, such as strength of the device as discussed below. Due to the longer chain length the strength, in particular the tensile strength, of the device is increased. The chain length of the first polymeric component is preferably above 100 monomer units, such as above 120 monomer units, preferably above 150 monomer units. The chain length of the second polymer is preferably at most 99% of the chain length of the first polymer, such as at most 95%, such as at most 90%, such as at most 80%, such as at most 70%, such as at most 60%, such as at most 50%, such as at most 40%, such as at most 30%, such as at most 20%, such as at most 10%, such as at most 5%.

In an embodiment the first polymeric component is selected from polymers having a carbon-backbone.

The first polymeric component may be selected from polyacrylates, polystyrene, polyethers, polytetrafluorethylene, polyvinylalcohol, polyethylene, polypropylene, polyolefinic polymers, polyethylene, polypropylene, polyethylene oxides, polyvinylpyrrolidone, polysilanes, polyurethanes, polyethers, polyamides, polyesters, polyalkyl acrylates, nylon, rubber and epoxy resins. It should be understood that the above list of polymers is not exhaustive, and other polymers may also be employed in the present invention. Preferred is polyethylene and polypropylene. Most preferred is polyethylene.

When the device is constituted by two polymeric components, each optionally comprising one or more metal components, the second polymeric component may be selected from polyacrylates, polystyrene, polyethers, polytetrafluorethylene, polyvinylalcohol, polyethylene, polypropylene, polyolefinic polymers, polyethylene, polypropylene, polyethylene oxides, polyvinylpyrrolidone, polysilanes, polyurethanes, polyethers, polyamides, polyesters, polyalkyl acrylates, nylon, rubber and epoxy resins. Preferred combinations for the first and the second polymeric component are polyethylene and polypropylene, polyethylene and polyethylene, or polypropylene and polypropylene, in the latter two cases, the first and the second polymeric components is comprised of identical monomers, whereas the polymers thereof are of different chain length. When the monomers of the two polymeric components are identical the prosthetic device is preferably compounded to form a bidispergent system.

The device is to be substantially composed of polymeric material optionally comprising one or more metal components, particularly solid or semi-solid polymers. Polymers are the family of synthetic or natural macromolecules consisting of inorganic, organic polymers and combinations thereof. Organic polymers may be natural, synthetic, copolymers, or semisynthetic polymers. Natural polymers comprise of the class of compounds known as polysaccharides, polypeptides, and hydrocarbons such as rubber and polyisoprene. Synthetic polymers comprise elastomers such as nylon, polyvinyl resin, polyvinyl chloride, polyvinyl dichloride, polyvinylpyrrolidone, polyethylene, polystyrene, polypropylene, polyurethane, fluorocarbon resins, acrylate resins, polyacrylates, polymethylmethacrylate, linear and cross-linked polyethylene, phenolics, polyesters, polyethers, polypyrolidone, polysulfone, polyterpene resin, polytetrafluoroethylene, polythiadiazole, polyvinylalcohol, polyvinylacetal, polyvinyl oxides, and alkyds. Semisynthetic polymers may be selected from cellulosics such as rayon, methylcellulose, cellulose acetate and modified starches. Polymers may be atactic, stereospecific, stereoregular or stereoblock, linear, cross-linked, block, graft, ladder, high, and/or syndiotactic. The term graft polymer is intended to mean copolymer molecules comprising a main backbone to which side chains are attached. The main chain may be a homopolymer or copolymer and the side chains may contain different inorganic or organic constituents.

The device may comprises cross-linked polymers elastomers such as high consistency elastomers, rubber, elastin and collagen. The material may be selected from polyurethane, elastin, collagen and combination products thereof. Alternative embodiments of materials suitable for the surface of a device according to the invention include, in addition to the materials mentioned supra and infra include hyaluronic acids and derivatives thereof.

Preferred polymeric materials are however presently believed to be those selected from the group comprising polyolefins, such as polyethylene, polypropylene, polybutene, polyisoprene, and polyvinylpyrrolidone, combinations thereof their copolymers, and grafted polymers thereof, particularly polyethylene and polypropylene, most particularly polypropylene. These polymers may be combined with metal, which is in a form described elsewhere herein.

Preferably, the polymer materials of the first, second and/or third polymer layer, each optionally comprising one or more metal components, may be from the group of polyethylenes or the group of polypropylenes such as polyethylene (PE), polypropylene (PP), high molecular weight polypropylene (HMWPP), high molecular weight polyethylene (HMWPE), ultra high molecular weight polyethylene (UHMWPE) and ultra high molecular weight polypropylene (UHMWPP), high density polyethylene (HDPE), low density polyethylene (LDPE), high density polypropylene (HDPP) and low density polypropylene (LDPP), ultra high density polyethylene (UHDPE), ultra high density polypropylene (UHDPP), cross-linked polyethylene, non-cross-linked polyethylene, cross-linked polypropylene, and non-cross-linked polypropylene. In this embodiment of the present invention, any combination of polymers listed above, or their equivalents, may be used. These polymers may be combined with metal, which is in a form described elsewhere herein.

In an embodiment the first, second and/or third polymeric component is a composite material. The device may be fully or partly fabricated of a composite material. This composite material may be a fibre-reinforced composite material comprising fibre embedded in a polymeric matrix. After embedding of a fibrous material in a polymeric matrix, the resultant fibre-reinforced composite material is shaped and is partially or fully cured to the point of sufficient hardness to provide a component for use in the fabrication of the medical device.

The reinforcing fibre element of the fibre-reinforced composite may be formed of one or more materials selected from the group consisting of: graphite fibre, polyaramid, polyesters, polyamides, nylon fibre, carbon fibre, glass fibre, collagen fibre, ceramic fibre, polyethylene fibre, poly(ethylene terephthalate), polyglycolides, polylactides, stainless steel fibre, cobalt-chrome alloy, titanium, titanium alloy, or nickel-titanium shape memory alloys, biocompatible polymeric materials, and other natural and synthetic materials compatible with the polymeric matrix.

The composite materials can also include reinforced plastics, or polymers which are laminated or layered or reinforced with one or more other materials such as nylon, graphite fibre, Kevlar.RTM. fibre, stainless steel fibre, etc., nylon fibre, carbon fibre, glass fibre, collagen fibre, ceramic fibre, polyethylene fibre, poly(ethylene terephthalate), polyglycolides, polylactides, and combinations thereof.

The polymeric matrix element of the fiber-reinforced and particulate-filled composites is selected from those known in the art of materials used for medical devices, including but not being limited to polyamides, polyesters, polyolefins, polyimides, polyarylates, polyurethanes, vinyl esters or epoxy-based materials. Other polymeric matrices include styrenes, styrene acrylonitriles, ABS polymers, polysulfones, poly-acetals, polycarbonates, polyphenylene sulfides, and the like. Other polymeric materials mentioned herein can also be used.

The reinforcing material may be in the form of a network of fibre formed of one or more materials as mentioned above, where the network is embedded in one or more polymers. The network or the fibres are embedded in a polymer, where the polymers are described elsewhere herein.

In the method of the present invention, the device is made after the embedding of the fibre with a polymeric matrix. After embedding of the fibre, the resultant composite material is formed into, for example, a long bar and cured or polymerized to a hardness whereby the bar may be cut and/or machined without deforming the structural integrity of the bar. The bar is preferably cut into short segments and is ready for use in the fabrication of medical devices. The bars may be used as they are or may be further modified by cutting, grinding, machining, heating and shaped and the like to provide a specifically shaped or customized component.

The medical device in accordance with the present invention is preferably formed from a fiber-reinforced composite material comprising a polymeric matrix and reinforcing fibre within the matrix. The fibre are embedded in the matrix manually or mechanically by a variety of techniques including, but not limited to matched die processes, autoclave moulding, resin injection moulding (RIM), sheet dough and bulk moulding, press moulding, injection moulding, reaction injection moulding, resin transfer moulding (RTM), compression moulding, open moulding, extrusion, pultrusion and filament winding.

It is further contemplated that the fibre or wires of metal can be interwoven with non-resorbable polymers such as nylon fibre, carbon fibre and polyethylene fibre, among others, to form a metal-polymer composite weave. Further examples of suitable non-resorbable materials include DACRON and GORE-TEX.

The fibre may further be treated, for example, chemically or mechanically etched and/or silanized, to enhance the bond between the fibre and the polymeric matrix. The fibre preferably take the form of long, continuous filaments, although the filaments may be as short as 0.1 to 4 millimeters. Shorter fibre of uniform or random length might also be employed. The fibrous element may take the form of a fabric. Fabric may be of the woven or non-woven type and is preferably preembedded with a polymeric material as set forth herein. The fibrous component may be present in the fiber reinforced composite material in the range from about 20% to about 85%, and more preferably between about 30% to about 65% by weight.

In an embodiment the first and/or second and/or third polymeric component is nano-fibre and/or a nano-structured composite. The nanofibre is nanofibre of any of the polymers mentioned elsewhere herein.

The composite polymeric material may be a nano-structured composites in the form of reinforced polymers with low quantities (<25% eg. less than 15% or less than 5%) of nano-metric sized clay particles or nano-metric sized particles of any other material mentioned herein.

In an embodiment the first and/or second and/or third polymeric component is cross-linked. The polymer is selected from any of the polymers mentioned herein. The combination of a polymer having a high chain length and a polymer having a shorter chain length, but being cross-linked provides a strong device yet having the resilient properties necessary for the device.

In an embodiment the first and the second polymeric component comprises the same monomeric component or the same components of the composite material. Optionally these polymeric components further comprise one or more metal components, which may be of different length in the two polymeric component.

The device and units may be designed not to interfere and to be non-invasive with regards to intra-articular components when the device is in the joint cavity by means such as a slit in the body of the device.

Moreover, non-interference of the intra-articular components may be achieved by a hole which runs through the body of the device; that is to say the device may comprise a hole through which intra-articular components may pass. When loading the device, the slits may serve to pass intra-articular components through the body of the device. The slits in this embodiment run from the periphery of the body of the device to the hole through which the intra-articular components pass after the device is implanted or loaded.

Typically, and to at least some extent, the device is adapted in its structure and/or material composition to alleviate conditions associated with worn cartilage by providing a spacer function and/or to exert pressure distribution in the joint when the joint is loaded and/or to provide at least part of the sliding/rotating movement of the joint by internal movement of at least part of the device.

It is also an object of the present invention to provide a method for non-invasive locking of a device within a joint. In addition, the method may be independent of use of cement or bony ingrowth of the device.

A still further object of the present invention is to provide a kit for use in the method for non-invasive locking of a device within a joint.

It is also an object of the present invention to provide a method for preventing damage between mating surfaces or articulating surfaces within a joint such as between the femoral head and the acetabulum of a hip joint.

A more specific object of the present invention relates to a prosthetic device for insertion into a joint cavity of a joint of a vertebrate such as a human, the device is being adapted to provide a spacer function and/or to exert stress distribution in the joint when the joint is loaded and/or to provide at least part of the sliding/rotating movement of the joint by internal movement in the material of at least part of the device, the device being capable of being fixed or retained in the joint cavity in a manner which is substantially non-invasive with respect to cartilage and bone natively present in the joint cavity.

When inserted into the joint cavity the device can be constructed to locking itself to an intra-articular component and thereby being fixed or retained in the joint cavity.

Physical-Structural Features of the Device

The physical-structural features of the device relate to the size, form or shape of the device as well as the structural components and design components of the device.

Size and Shape

The overall shape of the device is such that it substantially fits into the excising anatomical dimensions of the joint. In general, the size and shape of the device may be such that the device fits into the intra-articular cavity in that it may partially or fully occupy the space defined by the cavity, this depends on the type of device. For some of the joints it is preferred that the extent of the device, when positioned in the joint cavity, is larger than the normal extent of cartilage on the bone end in that joint. In an embodiment, a hole runs through the body of the device to allow intra-articular components to traverse the body of the device and thus be surrounded by the device.

In this embodiment the device may be construed in a liberal sense as essentially torus-shaped in that the device can be of a plurality of geometrical shapes, symmetrical and asymmetrical, comprising a hole which runs through the body to create an internal tubular passage through which intra-articular components may pass.

The device may also be ball-shaped, disc-shaped, spherical, globular-shaped, cup-shaped, cone-shaped, ring-shaped, cylindrical and have convex, concave, or flat surfaces. Accordingly, the body of the device shape can e.g. be in the form of a horseshoe, a curl, ring-shaped, circular or semicircular so as to be suitable for fitting into the anatomical dimensions of the particular joint. Furthermore, the device may be asymmetrical.

The body of the device may be of a geometrical shape comprising a surface having the form of body shaped by rotating a circle about a coplanar axis which does not intersect the circle. It may be ball-shaped, disc-shaped, globular-shaped, cup-shaped, cone-shaped, ring-shaped, cylindrical and may comprise convex, concave, or flat surfaces. In some aspects it is characterised in that it comprises a hole extending from one surface of the body of the device to the same or another surface, creating an internal tubular cylinder. This internal tubular cylinder may be straight if the hole extended to two parallel surfaces, curved if the hole extends to perpendicular surface, U-shaped if the hole extends to two parts of the same surface or a combination of one or more of these internal shapes and thus tortuous.

Certainly, given that the overall shape of the device is such that it substantially fits into the excising anatomical dimensions of the joint, it is anticipated that the body of the device may be asymmetrical or of no definable shape so as e.g. to the fill the intra-articular cavity, to allow for the movement of the intra-articular components during the flexing of the joint, to support intra-articular components or to support matter which form the walls of the cavity.

It is preferable that the shape of the device is such that it does not impede die normal functioning of the joint and its components.

It is particularly anticipated that the body of the device may be asymmetrical or of no definable or uniform shape when die device is for use in a hip joint. Alternatively, the shape of the device may be such that it resembles the native cartilage, or part thereof, naturally present in the joint cavity.

Accordingly, in the case of a hip joint, the shape of the device is preferably such that it fits into the existing space of the joint cavity comprising ligamentum capitis femoris, the “walls” of the space being defined by the concave shape of the acetabulum and by the convex shape of the femoral head.

Moreover, the overall shape of the device may be a result of an assembly of more than one units of the device, such as the assembly of two or more rings of different sizes stacked upon each other so as to form a cone-shaped device. The assembly of units may be done in vivo or ex-vivo.

Furthermore, in an embodiment, the overall shape is such that the device is capable of locking itself to an intra-articular component if present in the joint and thereby being fixed or retained in the joint cavity. When the intra-articular component is a ligament the shape is such that the ligament is surrounded or substantially surrounded by the device.

However, the overall shape of the device may have any other form as long as the material is of such a character that the device when present in sit fits into the joint cavity, for example due to elastical deformation of the device.

Preferably, the elastical deformation of the device is such that the presence of ligamentum capitis femoris results in a shape leaving room for the ligamentum. Otherwise, the surface of the upper part of the device facing the acetabular cavity may comprise a groove embedding the ligament.

Typically, the shape of the device is formed from a moulding of its materials or from a casting process. It may alternatively be the result of a framed structural construction or skeletal assembly. It is typically solid in that the body of the device is not hollow but rather such that the material of the device comprises all or essentially all of the space between two surfaces. The moulding, casting, construction or assembly may form a device into a uniform or non-uniform shape.

The device is essentially uniform in its stiffness or compressibility. However, when loaded, the material may have a tendency to deform in such a way that the locking mechanism is altered. This may occur if the element adapted to surround the ligament, when present in situ, has a slit which expands or gapes upon loading when the device is pressed together. This gaping may be further pronounced when the patient is e.g. walking whereby the ceiling of the acetabulum is pressed down on the upper surface of the device and the lower surface of the device is pressed down on the spherical surface of the femoral head.

Due to the rolling movement (rotation within the joint) of the femoral head, the possibility exists that the femoral head may press itself up into the slit of the device during the movement. In such cases, the press distribution and/or internal movement of the device may be limited to a minor part of the device that may result in an undesirable increased pressure on that portion of the device. Finally, contact between the femoral head and the acetabulum may occur in case the femoral head penetrates through the device. However, a device comprising parts overlapping each other can prevent this possible undesirable effect.

Accordingly, as mentioned above the device may be curl-shaped whereby the device with respect to the slit or opening has overlapping parts which do not represent a complete opening in the loading direction.

The size of the prosthetic device according to the invention may be of any size corresponding to the dimensions of the joint. In a hip joint a suitable size is normally one that allows the diameter of the device to be about the same or less than the diameter of the femoral head. However, on some occasions the diameter may exceed that of the femoral head. The size may also depend on the degree of damage of the native cartilage of the joint. Moreover, the space available within the joint in the individual may have an effect on the preferred diameter. Also the compressibility of the material should be taken into account. In the case in which the material is highly compressible, the device may increase in diameter upon loading of the joint when loaded, the device should generally cover the surface area which is covered with cartilage in the normal joint, e.g., in the hip joint, the surface of caput femoris should preferably be substantially covered when the joint is loaded to avoid contact of the surface of the femoral head with the acetabulum.

The length of the diameter of the device is designed to fit into the particular joint, such as between 1-80 mm, such as between 2-70 mm, preferable between 10-60 mm, more preferable between 15-50 nm, most preferred about 40 mm, when the joint is loaded.

The prosthetic device according to the invention may vary in thickness depending on the load on the joint, and the thickness of the device may also vary within the device.

The thickness of the device in each of the middle area and outer area is at least 0.1 mm, preferably between 0.2-60 mm, such as between 0.340 mm, preferably 0.6-30 mm, more preferably about 0.8-20 mm, most preferably about 1-15 mm in the unloaded stage. Depending on the material, the device may be highly compressible, whereby the initial thickness may exceed the above-mentioned upper limit. If only a limited rotation takes place in the joint, the thickness of the device may be decreased.

In one embodiment of the invention, the device is capable of locking itself to the intra-articular component by at least one element of the device surrounding the component in such a manner that displacement of the element and thereby the device, is limited by interlocking with the component. The intra-articular component which is surrounded is preferably a ligament, such as a ligament natively existing in the joint cavity.

In one embodiment of the device according to the invention, the element completely or substantially completely surrounds the ligament.

Thus, one embodiment relates to a prosthetic device according to the invention relates to a device wherein the element interlocking with a ligament when present in situ, permits the ligament to extend through the element and substantially exert its natural function on the joint.

In one aspect of the invention, the prosthetic device is intended for the articulation of a hip of a human, the device being adapted such that when present in situ in the human hip Joint cavity, it comprises at least one element surrounding ligamentum capitis femoris. Accordingly, ligamentum capitis femoris represents the surrounded intra-articular element mentioned above.

It is contemplated that the surrounding of the intra-articular component by the element may be a completely or substantially completely encircling of the ligament.

It is also preferred that the prosthetic device, when present in situ, comprises at least one ring-shaped or substantially ring-shaped element.

According to another aspect of the invention, the element of the prosthetic device which is adapted to surround the ligament when present in situ has such a shape and such properties that it can be placed around the ligament and stay interlocked with the ligament.

Structural Components

The device preferably comprises structural components which permit arrangement of the body of the device around native intra-articular components.

When the prosthetic device according to the invention is a hip endoprothesis, the device has a shape and structural components permitting arrangement of the body of the device around ligamentum capitis femoris.

A prosthetic device according to die invention comprises a device wherein the element of the device interlocking with the device with an intra-articular component has such a shape and/or properties that it is capable of replacing or supplementing worn or damaged cartilage in the joint and/or is capable of preventing wear of the native cartilage of the joint or of the bone tissue of the joint.

The structure of the material of the device or of a part of the device may be in the form of fibres and filaments of polymers and/or metal which can be incorporated into the matrix in a braided, woven, spongy or spiral pattern, the fibres and filaments having reinforcing properties. The polymer fibres may be inorganic fibres such as carbide, nitride, boride, carbon and oxide fibres, or the polymeric reinforcement may be of organic origin such as Dacron™. In a preferred embodiment the fibres are selected from polyethylene fibres, polypropylene fibres or a combination thereof. The metal may be any suitable metal e.g. titanium, gold, silver and/or chromium-cobaltum, and may be of any structure as described elsewhere herein.

The structure of the material of the device may comprise a layered or laminated structure, a core of one material or one or more interposed layers with different properties enabling an overall function of the devise suitable for providing a spacer function and/or to exert pressure distribution in the joint when the joint is loaded and/or to provide at least part of the sliding/rotating movement of the joint by internal movement of the device, or relevant part of the device. However, it is preferred that the material itself does not comprise interposed layers resulting in sliding between the layers and thereby tear on the mating surfaces within the device. Accordingly, the body of the device should be one continuous solid or semi-solid material.

In one preferred embodiment of the invention, the device comprises a tubular passage through which the ligament can pass and be surrounded by the body of the device. Circular movement around the substantially central ligament is possible but replacement of the device is prevented. A further feature of the structure of the device may be that of a slit extending from the outer surface of the device and through the body of the device into the central tubular passage. The slit may be curl-shaped in the radial direction with the axis of the tubular passage being the centre.

The slit may curl or curve into the body of the device so as to form an S-, or C-shaped slit, or zigzag or spiral slit. The curl of the slit may be in the to dimensions of a disc shaped device, or may curl in all three dimensions in the case of a globular, spherical, cone-shaped or cup-shaped device.

Furthermore, in embodiments where the device comprises more than one unit, the curvature of the slit may be such as to form a zigzag, spiral or S- or C-shaped multi-unit slit.

In multi-unit devices, the outer surfaces of the parts of the unit which are in contact with each other may have a surface pattern preventing the units from sliding apart such as grooves or etching or jagged surface pattern.

Moreover, the overall shape of the device may be from an assembly of two or more elements of one device, such as two semi-circular elements assembled to form a ring or from the assembly of two elements obtainable from the cross-sectioning of a ring or globular device along their longest axis. As was the case for the surface of two units, two elements may have a surface pattern preventing the elements from sliding apart such as grooves or etching or jagged surface pattern. Thus, a device and its shape may be the result of an assembly of two or more elements and/or two or more units, each comprising surfaces designed to preventing slippage of units and/or elements.

If suitable, the device may comprise a material which functions as a frame for the shape or secures the device from opening when placed in situ, for example in the form of a shaped component having the properties of a spring or the like.

In one embodiment, the ring-shaped body of the device has a slit or other suitable means which enables the device to be placed in the position encircling ligamentum capitis femoris.

Upon loading the device into the joint, the element of the device surrounding the component, e.g. a ligament, and thereby interlocking with the component, may tend to open up due to deformation of the device in the form of flattening resulting in an increased diameter. When the diameter of the device increases, e.g. the diameter of a ring-shaped device comprising a slit, the adjoining surfaces of the slit may gape.

As stated, during the compression, extension or rotation of de device when de device is present in a joint, the slit may have a tendency to gape and thus result in reduced weight-bearing effectiveness and/or result in trapping of intra-articular components within the seam of the slit. Preferably, the seam cannot be pulled apart in the direction of the plane of the seam by the mechanical pressure exerted by the body of the device conferred by the elastic properties of the material.

To prevent undesired slippage of the seam perpendicular to the plane of the seam, a variety of means may be incorporated into die design of the device so as to lock or adhere the two sides of the scam. Preferably, the locking or adherence means are reversible so as to allow removal or manipulation of the device after initial loading and use.

The seam is preferably characterised in that a smooth surface is formed in die plane of the seam.

To prevent die device from operating, the device preferably comprises overlapping or intersecting parts, such as lips or dovetails as is known by the person skilled in the art of mechanics or moulding. The two sides of the seam may be adjoined by means of an interlocking device such as a protrusion-hole device on sides of the seam. Alternatively, to prevent slippage in perpendicular to the plane of the seam, each side of the seam may be such that each side of the seam comprises an alternating sequence of angled grooves and corresponding extrusions. Moreover, the top and bottom portion of each side of the seam may comprise alternating teeth and sockets to prevent slippage. To prevent gaping such overlapping parts and their mating surfaces of the sides of the seam may have an interlocking surface structure. The pattern of such a structure may include depressions on the mating surface of one part and corresponding elevations on the other mating part of the device.

Accordingly, in one embodiment the overlapping parts are such that the interlocking surface structures constitute grooves. These grooves may extend radially, primarily resulting in a decreased tendency of the device to “open up” at the area corresponding to the slit or the gap. The grooves may also be orientated in a circulatory structure preventing the mating surfaces from gliding or sliding apart from each other. Additionally, the structure may comprise a combination of both elements reducing undesired movement in both of the two directions, when the device is deformed during loading of the joint.

The terms “radially” and “circular” should be understood as relative to the centre of the device or relative to the part of the device where the ligament extends through the device. “Radially” meaning e.g. grooves being located along radii from the centre, and the term “circular” meaning that e.g. the grooves are located along the periphery of a circle around the centre.

In another embodiment, the pattern includes other prominences or knobs, including pointed elevations. Thus, any structure comprising an elevation on one mating surface and a corresponding depression on the other mating surface may result in a decreased movement between the mating surfaces. Accordingly, any structure of the mating surfaces which thereby functions as an interlocking “hook” is within the scope of the invention. The mating surfaces of the curls may have an interacting profile in the form of a shape or pattern such as grooved surfaces which prevent the surfaces from sliding apart by reducing sliding movements between the mating surfaces upon loading of the device.

Another preferred embodiment of the invention relates to the seam created by the slit in the body of the device, accounts for preventing of slippage or gaping of the seam by means of a chemically treated surface of the sides of the slit. One embodiment of this aspect of the invention anticipates adherence of the two sides of the seam by means of photolytically or thermally activating a reaction between the chemically treated surfaces of the sides of the seam once the device has been loaded into the joint. Preferably, this adherence is reversible.

In another embodiment, the device may also comprise two or more separate rings each having a slit which are arranged so that the slits are orientated in such a way that no direct opening exists in the loading direction, accordingly, the slits are displaced in the direction parallel with the axis of the device. Mating surfaces of such rings may also have an interlocking structure as explained above.

In a still further embodiment the device is in the form of a curl, wherein the ring-shaped elements together have the overall shape of a cup. Also in this embodiment the mating surfaces may comprise grooves preventing sliding movements of the mating surfaces upon loading.

In a still further embodiment, the device may comprise minor vertical slits on the outer periphery of the device, these minor slits, e.g., having a depth of 1-5 mm may “absorb” the increasing diameter of the device upon loading. Preferably, the part of the device comprising the slits (the outer periphery) is not subject to heavy loading which could result in particulation of the edges of the device corresponding to the slits. These minor vertical slits on the outer periphery of the device may alternatively serve so as to not interfere with movable or immobile components of the joint within the cavity.

The device according to the invention may e.g. be processed by moulding of the material including extrusion and injection moulding. However, any other means for preparing the device of the desired shape could be utilised.

In addition, the device may comprise a dye or other material enabling visualisation of the device such as by X-ray.

Material Features

The material features of the device related to features conferred by the chemical composition of the device.

It is well known in the orthopedic field to use different types of materials for prostheses that are suitable for implantation in the body. The device may be produced from any material or combination of materials suited for implants. However, it is preferable that the body of the device does not comprise of any substantial extent of metallic materials.

The combination of materials can be varied according to the properties preferred for each device. However, for some implant types, the body of the device is constituted of polymeric material or materials optionally comprising one or more metal components.

Preferably, the material of which the device is made is biocompatible, e.g. hemocompatible, thromboresistant, non-toxic, and/or non-carcinogenic. In addition, the material should be resistant to particulation, and the solid surface of the material should be so that the surface tension is suitable for the interaction between the material and the biological surfaces.

Biocompatibility may be assayed through in vitro tests as well as animal tests. Enzymatic biodegradation may be used as indicative of biocompatibility. Furthermore, chondrocytes and fibreblasts may be grown on the material to evaluate the compatibility.

Finally, biocompatibility may be evaluated by implanting devices of the material in animals and examining the animal and/or device after a period of time.

Polymers and copolymers of polypropylene or polyethylene, as well as grafted forms of each of these are particularly interesting. Moreover, surface treated forms of these polymers, copolymers or grafted polymers are of notable interest.

The structure of the polymeric material of the device or of a part of the device may be in the form of fibres and filaments which can be incorporated into the matrix in a braided, woven, spongy or spiral pattern, the fibres and filaments having reinforcing properties. The fibres may be inorganic fibres such as carbide, nitride, boride, carbon and oxide fibres, or the reinforcement may be of organic origin such as Dacron™. In a preferred embodiment the fibres are selected from polyethylene fibres, polypropylene fibres or a combination thereof. The fibres may be surface treated before incorporated into the matrix to obtain a better adhesion of fibres to matrix.

The present invention in particular relates to a device composed of material formulations intended to meet the specifications of durability, biocompatibility, etc. These properties are obtainable by treating polymer materials, such as polyethylene, polypropylene or polyvinylpyrrolidone or combinations and co-polymers thereof as well as precursor materials for polymerisation, with high-energy electrons, gamma rays, photons, microwaves, ion implantation, plasma treatment, annealing, thermal radiation or another radiation to obtain ideal durability and biocompatibility of the new, modified material. Treatment of the above-mentioned materials with radiation leads to cross-linking of polymers and thereby generating new, modified materials. Preferably, the polymer material is a cross-linked polypropylene material. In another embodiment the polymer material is a cross-linked polyethylene material.

In one embodiment the device comprises a body constituted by the first and the second polymeric components, each polymeric component optionally comprising one or more metal components. The body may optionally be treated in order to optimise the properties such as surface properties, biocompatibility and/or low friction. By the term “body of the device” is meant the part of the device providing the strength properties as well as the resiliency properties.

In another embodiment the device comprises a body constituted by the first polymeric component, whereas the second polymeric component provides optimised surface properties.

Furthermore, radiation also allows grafting of polymers onto existing polymer surfaces, resulting in new mechanical properties as well as new surface properties. In is manner, the resulting modified polymer device can be processed to meet the necessary requirements of durability and biocompatibility.

Polymers may be prepared by methods known to the person skilled in the art. Chemical catalysis, thermal induction or photo induction are anecdotal non-limiting examples of methods of preparing the polymers. The cross-linking of the polymers or grafting may be done by radiation or other methods known to the person skilled in the art.

The properties of the materials to be obtained by these cross-linking and grafting processes are preferably i) resistance to tear and wear; ii) good compressibility; iii) flexibility and surface properties which will allow wetting with biological fluids, and/or eventually allow growth of chondritic cells onto the prosthetic device.

Typically, the device is prepared by a process comprising of the following steps:

The prosthetic devise may be formed by casting the pure polymer optionally comprising one or more metal components, or a blend of polymers optionally comprising one or more metal components in a mould of specified dimensions. The polymer is chosen from the above mentioned polymers. The metal component may be chosen from the metals mentioned elsewhere herein.

After hardening the cast material as formed, or after swelling in a suitable solvent the device is subjected to high-energy electrons, gamma rays or another radiation in order to create cross-linking which will modify the mechanical properties of the cast material to meet the preferred specifications.

Finally, eventually after removal of the swelling solvent, the surface of the cast material is treated to achieve good surface properties as described above.

The surface of the device can subsequently be treated to modify surface properties such as wetting ability and/or biocompatibility. This surface treatment can be performed by plasma treatment, chemical grafting or by a combination of plasma treatment and chemical grafting. The surface of the device contacting with the articulating surfaces of the joint may be of such a material which forms a uniform contact surface reducing the overall contact stress per unit are and thereby avoiding corrosion of the articulating surfaces of the joint. Accordingly, the material contacting with the biological surfaces may be smooth, biocompatible, preferably self-lubricating, and it should be wear-resistant so that powder generated due to wear is avoided in that this could otherwise result in foreign matter reactions and cause further trouble to the function of the joint.

Furthermore, the surface material should preferably be a material or a combination of materials having self-repairing properties so that fissures, cracks or other ruptures on the surface do not exceed uncontrollable levels. However, the surface material is preferably continuous with the material of the rest of the device, e.g. the material may gradually merge into the material of the inner core or matrix of the device.

The surface of the material may be chemically treated so as to soften, rigidity or lubricate the surface of the device or parts thereof. The surface of the material may be coated so that the coating confers these properties, or may be treated so as to chemically alter the surface of the device so as to confer any of these properties. Alternatively, certain polymer surfaces may be modified by means of thermal or photolytic energy.

Also the surface treatment may be provided by incorporating surface treatment polymer, such as polyvinyl pyrrolidone, into the matrix to maintain the good surface properties.

Independent of whether the body of the device comprises one or two components, it is preferred that the body of the device is provided with a treatment resulting in a functional surface of the device being wettable by the joint fluid normally present in the joint cavity, in order to decrease any friction between the device and joint parts, such as bone, cartilage, ligaments and mucosa.

Without being bound by theory it is also believed that a wetted surface reduces the risk of having the immune system recognising the device when implanted, which would otherwise lead to adverse effects of the device.

By the term “functional surface” is meant the external surface of the device, ie. the surface contacting joint cavity parts. Since the body of the device is often produced as one, two or even three dimensional networks, internal surface may be present in the body, the internal surfaces often corresponding with the external surfaces.

The prosthetic device may also comprises a third polymeric component, the third polymeric component being different from the first and/or the second polymeric component. The third component will preferably be grafted to the body of the device and result in the improved surface properties. The third polymeric component is preferably selected from polyethylene oxides, and polyvinylpyrrolidone, most preferably from polyvinylpyrrolidone.

When the body is comprised of one component, such as wherein the first polymeric component comprises a copolymer of polyethylene and polypropylene or wherein the first polymeric component is a crosslinked polymer, the second polymer may be grafted to the first polymer and act as the third polymeric component as described above. The first and/or second polymeric component may optionally comprises one or more metal components.

Preferred devices are composed of:

A body of polyethylene having polyvinylpyrrolidone grafted thereto

A body of two polyethylene polymers of different chain lengths having polyvinylpyrrolidone grafted thereto

A body of polypropylene having polyvinylpyrrolidone grafted thereto

A body of two polypropylene polymers of different chain lengths having polyvinylpyrrolidone grafted thereto

A body of a copolymer of polyethylene and propylene having polyvinylpyrrolidone grafted thereto

A body of a polyethylene and a copolymer of polyethylene and polypropylene having polyvinylpyrrolidone grafted thereto

A body of polypropylene and a copolymer of polypropylene and polyethylene having polyvinylpyrrolidone grafted thereto

A body of polyethylene having 2-vinylpyrrolidone grafted thereto

A body of two polyethylene polymers of different chain lengths having 2-vinylpyrrolidone grafted thereto

A body of polypropylene having 2-vinylpyrrolidone grafted thereto

A body of two polypropylene polymers of different chain lengths having 2-vinylpyrrolidone grafted thereto

A body of a copolymer of polyethylene and propylene having 2-vinylpyrrolidone grafted thereto

A body of a polyethylene and a copolymer of polyethylene and polypropylene having 2-vinylpyrrolidone grafted thereto

A body of polypropylene and a copolymer of polypropylene and polyethylene having 2-vinylpyrrolidone grafted thereto

In the list polyvinylpyrrolidone and 2-vinylpyrrolidone need not be grafted to the device.

Insertion

It is also an object of the present invention to provide a method for introducing a device according to the present invention into a joint. The method comprises:

a) locking the device to an intra-articular component and thereby fixing or retaining the device in the joint cavity in a manner which is substantially non-invasive with respect to cartilage and bone natively present in the joint cavity.

The method may further comprise any of the following steps before locking the device to the intra-articular component in the joint:

i) exposing the joint capsule by conventional surgery procedures,

ii) penetrating the joint capsule into the joint space leaving a passage for

iii) introducing the prosthesis into the joint capsule via the passage, the prosthesis having a shape suitable for being introduced through this passage.

Locking the device to the intra-articular component and thereby fixing or retaining the device in the joint cavity in a manner which is substantially non-invasive with respect to cartilage and bone natively present in the joint cavity may include encircling a ligament present in the joint with a ring-shaped element of the device such as a ring-shaped device having a slit extending from the periphery of the device to the central opening of the “ring”.

The method may her comprise the steps of deforming the prosthetic device into a reduced volume or a slender shape before locking the device to the intra-articular component.

In the case of insertion into a hip joint, the insertion of the device is preferably performed after penetration through the head of the rectus femoris muscle leaving a passage having a substantial width for introducing means into the joint capsule without alteration of the function of the capsule after the surgery.

Means or instruments for inserting the device into the joint space can be in the form of forceps comprising means for deforming the device into a minor volume or a more slender shape and may comprise means for grasping around the intra-articular component to which the device is capable of interlocking.

The forceps may further comprise means for locking the device around or substantially around the intra-articular component and optionally means enabling the forceps to be withdrawn without withdrawing the device.

Thus, a further object of the invention relates to a kit comprising:
a) an intra-articular prosthetic device for a joint having
a.1) a spacer function and/or capability to exert pressure distribution and/or sliding/rotating movement of the joint by an internal movement of the device by means of a resilient member, and
a.2) a locking mechanism adapted to fix the device to an intra-articular component by means of an element of the device surrounding the component in such a manner that displacement of the device is limited by interlocking with the component; and
b) an instrument for inserting the prosthetic device into a joint cavity.

Preferably, the elements of the kit should be sterile.

The instrument b) may further comprise one or more of the following means b.1 to b.4:
b.1) means for deforming the prosthetic device into a reduced volume or to a slender shape and keeping this volume or shape upon introduction of the device to the joint;
b.2.) means for grasping or encircling the intra-articular component to which the element of the prosthetic device is capable of interlocking;
b.3.) means for leaving the prosthetic device in the joint with the element of the prosthetic device surrounding an intra-articular component; and
b.4.) means for retracting the instrument from the joint.

It is contemplated that each of the means of b.1.), b.2.), b.3.) and b.4.) may be connected to or form part of a handle. Moreover, the resilient member of a.1) and the element surrounding the intra-articular component of a.2) may constitute the prosthetic device.

The means of b.2.) for grasping or encircling the intra-articular component may comprise an incision of the instrument which, in situ, is able to substantially retain the element within the “legs” of the incision.

Biological Activity of the Device

When inserted in the joint cavity the device is capable of alleviating the pain and other symptoms related to damaged cartilage, such as improving movements. Furthermore, the device may be capable of healing the sick bone's structure and/or cartilage structure—in hole of partly.

For example the device may facilitate creation of new cartilage and/or minimise destruction, such as fibrillation and/or fragmentation, of cartilage by relieving the pressure on the residual cartilage/bone in the joint

Furthermore, the device may comprise biological active additives. Medication or biological active substances can be used as additive to the device to facilitate healing, minimise destruction or with other therapeutic goals, such as pain relieve, anti-inflammation, oncology treatments, simulation of bone growth, and/or anti-infectious agents. Also, biological osteogenic or chondrogenic, chondral inductive, and/or chondral conductive materials may be added to the device. In particular patients suffering from osteoporosis or other bone degenerating conditions may benefit from having devices comprising osteogenic inductive materials implanted.

The device itself can be used as a growth medium and/or network for the natural or artificial cells, such as chondrocytes.

The device is capable of being formed in the production process to suit any joint cavity of animals or human beings, therefore the device may for example be formed to fit into any one of the following Joints: Hip joint, knee joint, ankle joints, shoulder joint, elbow joints, wrist, fingers, spinal column joints, such as for substituting intervertebral discs, and the jaw joint.

The material for the medical devices is primary polymers, with at least one layer of a first polymeric component with high molecular weight, and at least a layer of a second polymeric component with low molecular weight. This combination of longer and shorter polymers provides the feature of the device comprising strength as measured by tear, tension and compression. These polymers may optionally comprise one or more metal components which is described elsewhere.

In one of the invention a medical device comprises a bio-compatible polymeric product with a layered structure comprising at least one upper layer of a first polymeric component, a middle layer of a second polymeric component, and at least one lower layer of a third polymeric component, wherein the chain length of the first polymeric component and the third polymeric component is longer than the chain length of the second polymeric component. These polymers may optionally comprise one or more metal components which is described elsewhere.

An important step of the present invention is the selection of the composition of the different polymer layers optionally comprising one or more metal components, as well as the selection of the number of polymer layers comprising first, second and third polymeric components, as well as thickness of the polymer layers and also size and position of an optional layer of the second polymeric component. These polymers may optionally comprise one or more metal components which is described elsewhere.

Another embodiment of the present invention provides for layers of polyolefinic polymers and resins. Within the context of the present invention, a polymer is defined as an organic compound having repeating units of similar or different monomers. A resin is defined herein as a partially cured polymer having utility as a moldable material suitable for curing into a solid article.

First Polymeric and Third Polymeric Component

The polymers comprising the first polymeric component and the third polymeric component may be above 100 monomer units, such as above 1,000 monomers units, for example above 10,000 monomer units, preferable above 20,000 monomer units, more preferable above 30,000 monomer units, further preferable above 40,000 monomer units, yet further preferable above 50,000 monomer units, most preferable above 60,000 monomer units.

The polymers of the upper and lower layer comprising first and third polymeric components of the present invention have preferably molecular weights ranging between 1,000 and 100,000,000 such as between 10,000 and 75,000,000, for example between 50,000 and 50,000,000, preferable between 75,000 and 25,000,000, more preferable between 100,000 and 1,000,000, further preferable between 200,000 and 800,000, yet her preferable between 300,000 and 700,000 most preferable between 400,000 and 600,000

In a preferred embodiment the polymers comprising the first polymeric component and the third polymeric component are comprises long polymer fibres, filaments or stands produced from the polymers presented above, preferred polymers to produce the fibre and filaments may be selected from the group of poly-ethylenes including, but not limited to, high molecular weight polyethylene (HMWPE), ultra hi molecular weight polyethylene (UNRIPE), high density polyethylene (HDPE), ultra high density polyethylene (UHDPE), cross-linked polyethylene and non-cross-linked polyethylene. The most preferred polymer of the invention is fibre produced from UHMWPE. The polymers of the first polymeric component and the third polymeric product provide strength and wear resistance to the device.

A preferred polymer of the upper and lower layer of the invention is UHMWPE, and a preferred combination is UHMWPE and HDPE.

Additional components of the upper and lower layer polymeric material may be incorporated into the matrix in a braided, woven, spongy or spiral pattern, the fibres and filaments comprising the additional components having reinforcing properties. The fibres may be inorganic fibres such as carbide, nitride, boride, carbon and oxide fibres, or the reinforcement may be of organic origin such as Dacron.

The first and third polymeric component may also be any other polymeric component described elsewhere herein.

The Second Polymeric Component

The middle layer comprising a second polymeric component can be constructed from short chain polymer material; the polymers may be selected from the polymers presented above. Short chain polymers may have less than about 100 units, such as less than about 90 units, for example less than about 80 units, preferable less than about 70 units, more preferable less than about 60 units, further preferable less than about 50 units, yet further preferable less than about 40 units, most preferable less than about 30 units. The short chain polymer material may not have cross links and only weak Van der Waals forces between chains, The molecular weight is preferably less than about 10,000, such as less than about 9,000, for example less than about 8,000, preferable less than about 7,000, more preferable less than about 6,000, further preferable less than about 5,000, yet further preferable less than about 4,000, most preferable less than about 3,000.

Preferred polymers to produce the middle layer constituting film, core and inlay polymer layers may be selected from the group of poly-ethylenes or from the group of polypropylenes including, but not limited to polyethylene (PE), polypropylene (PP), high molecular weight polyethylene (HMWPE), high molecular weight polypropylene (HMWPP), high density polyethylene ADDLE), high density polypropylene (HDPP), low density polyethylene (LDPE) and low density polypropylene (LDPP). Preferred is short chain polymer material such as LDPE and LDPP. Further preferred are polymers which are branched. Most preferred is short chain polymer material of polyethylene.

From the above mentioned first, second and third polymeric components different polymer layers comprising fabric, fin, core and inlay are constructed. These polymer layers are further described below.

The second polymeric component may also be any other polymeric component described elsewhere herein.

Metal

The metal or metal components used in the devices may be any suitable metal which is biocompatible.

In a preferred embodiment the metal component is selected from one or more of the metals titanium, gold, silver, chromium-cobaltum, zirconia, cobalt-chromium-molybdenum alloy and/or a ceramic of one or more of these metals and alloys and may be of any structure as described elsewhere herein.

More preferred are titanium, gold, silver and/or chromium-cobaltum,

The metal components of the device may be in the form of fibre or filaments, although in this text “fibre” means both fibre and filaments. The metal components may also be in the form of powder, which in this text means powder or pellets or chopped fibres.

The Cobalt-Chrome alloys may be with the base metals cobalt and chrome mixed with smaller quantities of other metals.

The amount of cobalt in a cobalt-chrome alloy may be at least 25%, such as at least 30%, such as at least 35%, such as at least 40%, such as at least 45%, such as at least 50%, such as at least 55%, such as at least 60%, such as at least 65%.

The amount of chrome in a cobalt-chrome alloy may be at least 10%, such as at least 15%, such as at least 20%, such as at least 25%, such as at least 30%, such as at least 35%, such as at least 40%, such as at least 45%, such as at least 50%, such as at least 55%, such as at least 60%, such as at least 65%.

Any combination of the amounts mentioned above of cobalt and chrome which in total do not exceed 100% may be used as a metal in a device according to the present invention.

Preferred is an alloy with at least 34% cobalt at least 29% chrome mixed with smaller quantities of other metals. Optionally nickel may be one of the other metals.

Preferred is the alloy named Vitallium e.g. in the combination of 60% cobalt 20% chromium, 5% molybdenum, and traces of other substances.

Titanium alloys may be used as part of a device, in such alloys the base metal is titanium.

Preferred is a titanium alloy with aluminium. The aluminium amount may be 1-20%, e.g. 1-10%, such as about 4%.

Stainless Steel alloys may be used as part of a device, in such alloys the base metal is iron, mixed with larger quantities of chrome and nickel and some other metals

Preferred is a stainless steel alloy with iron in an amount of at least 40%, such as at least 50%, such as at least 60%, such as at least 70%.

Preferred is a stainless steel alloy with at least 58% iron, mixed with larger quantities of chrome and nickel and some other metals.

The metal can be used in any form, like powder, granulate, chopped fibres, long fibres, 2D structural components like plates, 3D structural components like shaped plates or Semicircles with holes. Also a combination of these forms may be used.

In different areas of a device different metals and/or different alloys may be used, e.g. in a layered structure comprising three polymer layers each further comprising one or more met component, each polymer layer may comprise e.g. fibers etc. of different or similar metal or alloys.

Fabric

From the longer polymer fibre comprising the first and/or the third polymeric component as previously described a fabric may be constructed. Optionally this fabric may also comprise metal fibre.

A fabric may constitute the upper and lower layers of a medical device.

The second polymeric component, optionally comprising one or more metal components, may also be in the form of a fabric. Preferred is a fabric of UHMWPE fibre. The fabric corresponds to the first and/or third polymeric component as described elsewhere herein.

Within the fabric, the first polymeric component and the third polymeric component are preferably in the form of fibre. Methods of construction of fibres are known to persons skilled in the art. The polymers may be aligned and/or spun into fibre by gel spinning or filaments, which again may be spun into strands. From the fibres and/or filaments and/or strands the layers of polymeric materials may be manufactured.

Metal fibre may also be treated to produce filaments and/or strands. The methods are known to persons skilled in the art.

The fabric may be produced into a suitable shape, the shape is preferably constructed by weave, knit, crochet, stitch, plait interlace, intertwine, interlock, link or unite the fibre and/or filaments and/or strands of polymeric and/or metal fibre in other ways such as non-woven techniques. Preferable the fabric is woven or knitted.

In an embodiment the fabric can be woven using different techniques, the techniques include but are not limited to cord woven, linen woven, mat woven, Celtic woven and twill woven. Persons skilled in the art know variations of these techniques, the variations is hereby incorporated.

According to an embodiment of the invention the polymer and/or metal fibres are woven into a squared fabric comprising intercepts with angles of 90 degree. The dimension and weaving style of the fabric is optional, preferred is a binding style of 3:1 (twill). The fabric can if the thickness allows it be rolled into a roll, from which suitable pieces are detached before the stratified polymer product is constructed. Products which can be used comprises but is not limited to fabric of Dyneema® from DSM, Spectra® from Allied Signal Inc. Preferably the fabric is workable in the process of construction of the medical device as described elsewhere herein.

In another preferred embodiment the fibres, filaments or strands of the constitution described above are woven into the fabric in a shape suitable for the shape of the polymeric product. The shape of the fabric can be any possible shape including but not limited to round, oval, triangle, quadrangle, square, rectangular, pentagon, hexagonal etc. and may be symmetrical or asymmetrical in any direction. Preferred shapes of the fabric are quadrangle and round.

In one embodiment the polymer and/or metal fibres in each layer of the fabric are positioned over each other making a structure wherein the angles of the intersect are of 1 to 179 degree, such as in angles of 40 to 150 degree, for example such as in angles of 60 to 130 degree, such as in angles of 70 to 110 degree, for example such as in angles of 80 to 100 degree, such as in angles of about 90 degree. Most preferred is intersects of fibre and strands in angles of about 90 degree.

The thickness of the fabric Is preferably determined by thickness as well as the number of fibres and/or filaments and/or strands and the distance between these fibres, filaments and strands in the fabric. The overall thickness of the fabric is preferably between 0.001 mm and 3 mm, preferred is between 0.01 mm and 2 mm, more preferred is between 0.02 mm and 1.5 mm, further preferred is between 0.03 mm and 1.0 mm, yet further preferred is between 0.04 mm and 0.08 mm, most preferred is between 0.05 mm and 0.06 mm.

In an embodiment the area weight of the fabric is preferred between about 10 g/M² and 500 g/M² preferred is an area weight of between about 50 g/M² and 300 g/M², more preferred is an area weight of between about 75 g/M² and 250 g/M², Per preferred is an area weight of between about 100 g/M² and 200 g/M², yet more preferred is an area weight of between about 125 g/M² and 175 g/M², even more preferred is an area weight of between about 140 g/M² and 160 g/M², most preferred is an area weight of about 150 g/M².

In an embodiment the thickness and/or area weight of the fabric varies across a single sheet of fabric. Hereby the area weight of the fabric also varies in the device. The area weight of a single fabric vary at least 5% across the fabric sheet, such as 10%, such as 20%, such as 30%, such as 40%, such as 50%, such as 60%, such as 70%, such as 80%, such as 90%, such as 100%, such as 120%, such as 140%, such as 160%, such as 180%, such as 200%, such as 250%, such as 300%, such as 350%, such as 400%, such as 450%, such as 500% according to the area weight of the thinnest 1 cm² of the fabric.

In an embodiment the thickness of the fabric has a first thickness in the middle area of the device and a second thickness in at least a part of the outer area of the device. The first thickness of may be smaller than the second thickness, and can vary as described above.

The fibres, filaments and strands from which the fabric is produced according to the description herein, may have a fibre diameter preferably between 100 and 650 dtex. The fibre diameter of the warpyarn is preferably about 300-650 dtex, more preferably about 350-550 dtex, further preferably about 400-500 dtex, most preferably about 430-460 dtex. The weftyarn is preferably about 100-350 dtex, more preferably about 150-300 dtex, further preferably about 175-250 dtex, most preferably about 210-230 dtex.

The fabric need not be constructed of fibre or filaments or strands with equal thickness. A woven fabric where some of the strands have a larger thickness than the rest may be used. In this way e.g. every second, every third or more strands in between may have a larger thickness than the rest of the strands of the fabric.

The fabric described herein may also be constructed by strands of different polymers and/or of different metals. The different polymers and/or of different metals may be selected among the polymers and metals listed herein above. Two or more polymers and/or metals may be used in the construction of the fabric.

In an embodiment the thickness of the fabric may vary according to different thickness of the polymer and/or metals strands as described above or different polymers and/or metals may be utilised to construct the fabric. Also different numbers of strands pr cm may be used.

In an embodiment the surface dimension of one or more inner layers of fabric may be smaller than the total surface dimension of a medical device. Smaller layers of fabric may enclose inlays.

In another preferred embodiment the fabric has a high tensile strength and a high wear resistance. The degree of tensile strength is determined by the polymer utilised to produce the fibre and the thickness of the fibre. The tensile strength of the strand or fibre in a fabric is preferably above 1.0 GPa, such as above 1.2 GPa, preferable above 1.4 GPa, more preferable above 1.6 GPa, further preferable above 1.8 GPa, yet further preferable above 1.9 GPa, most preferable above 2.0 GPa.

In another embodiment the tensile strength of the strand or fibre in a fabric is preferably above 0.05 GPa, such as above 0.1 GPa, preferable above 0.3 GPa, more preferable above 0.5 GPa, further preferable above 0.7 GPa, yet further preferable above 0.8 GPa, most preferable above 0.9 GPa.

Although the term ‘fibre’ is used in the description of fabric comprising the first and third polymeric components, filaments and/or strands and/or other components comprising long chains of polymer units may be used instead of fibres.

The fabric constitutes a reinforcement fabric or tissue of the device.

Film, Core and Inlay

If the device of the present invention is constructed as a laminated device, the middle layer of the polymeric product may comprise a second polymeric component optionally further comprising one or more metal components. The polymeric component optionally comprising one or more metal components may be any short chain polymer material or low density polymer material as described above. Also chopped strands of long chain polymer material such as fibre and/or filaments and/or strands may be utilised as short chain polymer materials. Preferred is when the chopped strands comprising short chain polymers are moulded into a matrix with low density polymer material or a polymer comprising the second polymeric component as described elsewhere herein.

By ‘chopped strands’ is meant shorter chains or strands cut from fibres and/or filaments and/or strands.

In an embodiment the middle layer comprising polymer layer comprises a film, a core or an inlay.

The polymer layers ‘film’, ‘core’ and ‘inlay’ may be produced of similar or substantially similar or different polymers optionally comprising metal components. Preferred are polymer layers of film, core and inlay which are produced by similar polymers optionally comprising metal components. Polymers suitable to be used are described above.

The differences of film, core and inlay may be the dimensions of the polymer layers. The dimensions are determined according to the function of the polymer layers. The film, core and inlay may differ in thickness from each other, but may also have similar thickness, whereby film and core sometimes can substitute each other in the composition of the medical device.

The visual difference of film and core is preferably based on the thickness, where the film in general is thinner than the core. The main purpose of a film layer is to attach two layers of fabric to each other, and simultaneously provide the device with characteristics such as capability of absorbing shocks, impacts and pressure load.

The core may also attach fabrics to each other, and provide the same characteristics to the device as the film, but the core may be utilised in devises subjected to higher degree of impacts and pressure load than to devises comprising no core layer.

The difference of core and inlay may be based on the length and width of the polymer layers optionally comprising metal components, the inlay may be smaller than a core. The function of an inlay is to absorb shocks and pressure in specific areas of a medical device. An inlay of one device may be larger than a core of another device.

In an embodiment the middle layer comprises a film or core or inlay. The film and core comprises the polymers described above optionally comprising metal components, and may be constructed by melting the polymers optionally comprising metal components. Mixtures of polymers may be used to construct the film, core or inlay. The melted polymeric mass may be formed according to any method possible, the methods are known to persons skilled in the art. The methods comprises but are not limited to blow moulding, extruding, foil moulding, injection moulding, compression moulding, preferred is blow moulding. Preferred methods are moulding of the melted polymeric mass in small or large open moulds/vats or injection moulding, the thickness of the material is optional, but is chosen not to be changed followed solidification. Following solidification the solidified polymeric matrix optionally comprising metal components can be cut or punched or stamped out to a suitable dimension.

The suitable dimension of the film may be determined in accordance to the scope of the application. The preferred application of the film is as a thin polymer layer optionally comprising metal components between two layers of fabric, in this situation the size comprising length and width of the film is at least the length and width of the polymeric material used to produce a medical device, hereby the film may be squared, circular or any other dimension as any surplus of polymer material is removed following formation of the medical device.

In an embodiment the device is constructed from layers of fabric, film, core and/or inlay where the layers each has a dimension suitable to construct the device without any process of removing surplus of polymer layers. In this process the polymer layers of film, core and/or inlay may have dimensions smaller than the outermost layer of fabric. To adjust the size of the polymer layers to the form of the device to be produced, inner layers of fabric may be smaller than the outermost layer of fabric. The outermost layer of fabric which constitutes the inner side of a medical device may also be smaller than the outermost layer of fabric which constitutes the outer side of a medical device.

The suitable dimension of the core may also be determined in accordance to the scope of the application. The preferred application of the core is as a polymer—optionally comprising metal components—layer between two layers of fabric, where the core fills in all the area comprising length and width between the two layers of fabric, in this situation the size of the core is at least the length and width of the polymeric material used to produce a medical device, hereby the core may be squared, circular or any other dimension as the surplus of polymer material is preferably removed following formation of the medical device.

The suitable dimension of the inlay may also be determined in accordance to the scope of the application. The preferred application of the inlay is as a polymer layer optionally comprising mew components, which fills in part of the area between two layers of fabric or film; hereby the inlay may comprises any dimension appropriate for the purpose of the medical device. The inlay is moulded into the appropriate dimension or it is cut into the appropriate dimension.

The preferred thickness of the core and of the inlay is chosen in accordance with a reduction of the thickness in the construction of the device. During the pressing process the thickness of the core and the inlay may be reduced by up to 50%, as the short chain polymers of the inlay and/or of the core are pressed in between layers of fabric. By this pressing process the other dimensions comprising length in two dimensions of the core and inlay may increase as the thickness decreases.

Objects of non-polymeric material or objects of polymeric material different to the material which the core or inlay is made of may be placed within core or inlay. The objects may be but is not limited to metal globes or metal sheets. The objects may be incorporated in the inlay or core in the moulding process or may be placed in holes made in the inlay or core in the moulding process or made afterwards. An example of objects in an inlay is metal globes in the inlay. These objects are different in composition and/or material type than the metal components described above used in the production of the core or inlay.

The difference of film and core has a fluid borderline, whereby the utility of film and core may be interchangeable. Also the difference of core and inlay has a fluid borderline, whereby the utility of core and inlay may be interchangeable

In an embodiment the film is prepared as described elsewhere, the film is preferably between 0.001 and 5 mm thick, such as between 0.01 and 5 mm, preferable between 0.1 and 4 mm, more preferable between 0.2 and 3 mm, further preferable between 0.3 and 2 mm, yet further preferable between 0.4 and 1.5 mm, most preferable between 0.5 and 1 mm.

The core or inlay which are also prepared as described elsewhere, is preferably between 0.1 and 30 mm thick, such as between 0.2 and 25 mm, preferable between 0.3 and 21 mm, more preferable between 0.4 and 17 mm, further preferable between 0.5 and 13 mm, yet further preferable between 0.6 and 10 mm, most preferable between 0.7 and 7 mm.

In an embodiment the surface dimension of one or more layers of film may be smaller than the total surface dimension of a medical device. Smaller layers of film may be used on one or more sides of smaller size fabric.

In a preferred embodiment the polymers as described above are of medical grade.

The film, core and inlay may comprise short chain polymers. Examples of characteristics, properties and additives of the short chain polymers are shown in the following tables

| | Method | Unit | Value |
|---|---|---|---|
| Characteristics | | | |
| Melt index | ISO 1133 | G/10 min | 0.8 |
| Density | ISO 1183 | G/cm³ | 0.924 |
| Property | | | |
| Yield Stress | ISO R527 | Mpa | 12 |
| Tensile Strenght at break | ISO R527 | Mpa | 14 |
| Elongation at break | ISO R527 | % | 650 |
| Modulus of elasticity | ISO 527-2 | Mpa | 240 |
| Melting point | ISO 11357-3 | ° C. | 114 |
| Vicat temperature | ISO 306 | ° C. | 98 |

Mechanical properties measured on a moulded plaque

More preferred examples of characteristics, properties and additives of the short chain polymers are shown in the following tables

| | Method | Unit | Value |
|---|---|---|---|
| Characteristics | | | |
| Melt index | ISO 1133 | G/10 min | 2.18 |
| Density | ISO 1183 | G/cm³ | 0.922 |
| Property | | | |
| Yield Stress | ISO R527 | Mpa | 11 |
| Tensile Strenght at break | ISO R527 | Mpa | 10 |
| Elongation at break | ISO R527 | % | 550 |
| Modulus of elasticity | ISO 5527 | Mpa | 210 |
| Melting point | ISO 11357-3 | ° C. | 112 |
| Melting point | ISO 11357-3 | ° C. | 94 |
| Shore hardness | ISO 868 | | 52 |

Mechanical properties measured on a moulded plaque.

Processing may be performed at an advised temperature of 150° C. to 180° C.

Additives may be used in the short chain polymer material, preferred is none slip agent and none anti-blocking additives.

An example of a short chain polymer material is Lacqtene® FE 8000 from Atofina.

The polymeric material of the film, core or inlay may include polymeric and/or metal fibres with a uniform length or with varying length. The fibres can be nano-fibres or chopped fibres. The material can also be a composite material e.g. of the type as described elsewhere herein. Further the material can be armoured polymers. In a nano-structured composite short and/or long composite material can be utilised.

The film, core or inlay may be produced by moulding, such as injection moulding, or injection extrusion. By controlled injection extrusion it is possible to control the crystal number and placing.

Production of a Layered Polymeric Product

In one embodiment the polymeric product optionally comprising one or more metal component from which medical devices are constructed comprises polymer layers in a sandwich or laminated format with at least three polymer layers, where the polymer layers are fused together by a heating process. Each of the polymer layers may optionally comprise one or more metal component. The middle or at least one inner layer may differ from the two or more outer layers, hereby the polymer layers may constitute a film, or a core or an inlay with at least one layer of fabric on each side. The fabric provides a high wear resistance and high tensile strength, while the inlay and core and to some degree also the film absorbs shocks.

In the production of a medical device, the different polymer layers as described above may be laminated in accordance to the required characteristics of the medical device. In an embodiment the middle or at least one inner layer of the polymeric product may constitute a core or a film, on each side of the core or film a fabric is positioned. In a preferred embodiment the polymeric product are composed of three layers where the fabrics at the different sides of the core have equal constitutions. Each of the polymer layers may optionally comprise one or more metal component.

The layers of fabrics within a device can be different according to the polymers and/or the metals utilised to produce the fabrics or the fibres, or strands within the fabrics may be different, or the fabrics are produced in different ways, also the fabrics can have different thickness. In a cup shaped device the outer part of the cup may comprise a ticker fabric than the inner part of the cup, hereby increasing the wear resistance of the outer part.

In another embodiment the polymeric product are composed of more than three layers, where, in between two fabrics a film or a core or an inlay are positioned. The individual layers of fabric may be substantially identical, identical or different in composition. Also the layers of film, core and inlay may be substantially identical, identical or different in composition.

In an embodiment the number of the layers core, film, inlay and fabrics differ across the polymeric product. The number of the layers can also vary In different areas of the polymeric product. The outermost layer of each side of the product must be a fabric, and two layers of fabrics have a film or a core or an inlay in between. With varying number of layers across the polymeric product, also the thickness of the product varies. Some areas may contain an inlay other areas may be without the inlay.

In a further embodiment the outermost layer of the product can be a film. The film is positioned next to a layer of fabric. Both sides of a product may have film layers as the outermost layers or only one side is a film layer. In case the medical device has more than two outer sides, one or more sides may be a film layer.

The polymeric product optionally with one or more metal components can also constitute two or more layers of fabrics on each side of a core or an inlay, and the two or more layers of fabrics may have a film of a polymer layer optionally with metal in between each fabric. Following heating of the layered polymeric product the film and/or core and/or inlay mechanically connect or bond together two layers of fabric.

Layers of film and/or core and/or inlay in a device may be different according to the polymers utilised to produce the layers. The polymers may be of different types, preferred polymers are mentioned herein above. Also the polymers may be mixtures of different types of polymers or mixtures of polymer chains of different length or both. Each of the polymer layers may optionally comprise one or more metal component.

Film may have a higher adhesiveness than core and inlay.

The number of layers of fabric in a medical device is optional, as well as the number of layers of film and fabric and inlay can differ on each side of a core or of an inlay. The number of layers of fabric in a medical device is preferably between 1 and 100, such as between 2 and 50, for example between 2 and 40, preferable between 2 and 35, more preferable between 2 and 30, further preferable between 2 and 25, yet further preferable between 2 and 20, most preferable between 2 and 10.

Also the number of layers of film in a medical device is optional, the number of layers of film is preferably between 0 and 100, such as between 1 and 50, for example between 1 and 40, preferable between 1 and 35, more preferable between 1 and 30, further preferable between 1 and 25, yet further preferable between 1 and 20, most preferable between 1 and 10.

In addition the number of layers of core in a medical device is optional, the number of layers of core is preferably between 0 and 100, such as between 1 and 50, for example between 1 and 40, preferable between 1 and 35, more preferable between 1 and 30, further preferable between 1 and 25, yet further preferable between 1 and 20, most preferable between 1 and 10. The number of layers of film, inlay and fabric can be different at each side of a core.

The number of layers of inlays in a medical device is optional, the number of layers of inlay is preferably between 0 and 100, such as between 1 and 50, for example between 1 and 40, preferable between 1 and 35, more preferable between 1 and 30, further preferable between 1 and 25, yet further preferable between 1 and 20, most preferable between 1 and 10. The inlay can be positioned anywhere within the stratified polymer product between two layers of film or fabric. The inlay may be smaller than the entire area of the medical device, and the inlay may be located at any position within the medical device. Also the number of layers of film, core and fabric can be different at each side of an inlay.

Preferred layered compositions of the polymeric product of medical devices comprises but are not limited to the constitutions:
fabric-film-fabric.
fabric-core-fabric.
fabric-film-core-film-fabric
fabric-film-fabric-core-fabric-film-fabric.
fabric-film-fabric-film-fabric-core-fabric-film-fabric-film-fabric.
fabric-film-fabric-film-fabric-film-fabric-core-fabric-film-fabric-film-fabric-film-fabric.
fabric-film-fabric-film-fabric-film-fabric-film-fabric-core-fabric-film-fabric-film-fabric-film-fabric-film-fabric.
fabric-core-fabric-film-fabric-film-fabric.
fabric-film-fabric-core-fabric-film-fabric-core-fabric-film-fabric.
fabric-film-fabric-film-fabric-film-fabric-core-fabric-film-fabric.
fabric-film-inlay-film-fabric.
fabric-film-fabric-film-inlay-film-fabric-film-fabric.
fabric-film-inlay-film-fabric-film-fabric-film-fabric.
fabric-film-inlay-film-fabric-film-inlay-film-fabric.
fabric-filmn-inlay-film-fabric-film-fabric-film-inlay-film-fabric.
fabric-film-inlay-film-fabric-film-inlay-film-fabric-film-fabric.
fabric-film-inlay-film-fabric-film-inlay-film-fabric-film-inlay-film fabric.
fabric-film-inlay-film-fabric-film-fabric-film-inlay-film-fabric-film inlay-film-fabric.
fabric-film-inlay-film-fabric-film-fabric-film-fabric-film-inlay-film fabric-film-inlay-film-fabric.
fabric-film-inlay-film-fabric-film-inlay-film-fabric-film-inlay-film-fabric-film-fabric.
fabric-film-fabric-inlay-fabric-film-fabric.
fabric-film-fabric-filn-fabric-inlay-fabric-film-fabric-film-fabric.
fabric-film-fabric-inlay-fabric-film-fabric-film-fabric-film-fabric.
fabric-film-fabric-inlay-fabric-film-fabric-film-fabric-inlay-fabric-film-fabric.
fabric-film-fabric-inlay-fabric-film-fabric-film-fabric-film-fabric-inlay-fabric-film-fabric.
fabric-film-fabric-inlay-fabric-film-fabric-film-fabric-inlay-fabric-film-fabric-film-fabric.
fabric-film-fabric-inlay-fabric-film-fabric-film-fabric-inlay-fabric-film-fabric-film-fabric-inlay-fabric-film-fabric.
fabric-film-fabric-inlay-fabric-film-fabric-film-fabric-film-fabric-inlay-fabric-film-fabric-film-fabric-inlay-fabric-film-fabric.
fabric-film-fabric-inlay-fabric-film-fabric-film-fabric-film-fabric-film-fabric-inlay-fabric-film-fabric-film-fabric-inlay-fabric-film-fabric.
fabric-film-fabric-inlay-fabric-film-fabric-film-fabric-inlay-fabric-film-fabric-film-fabric-inlay-fabric-film-fabric-film-fabric.
film-fabric-film-fabric-film.
film-fabric-core-fabric-film.
film-fabric-film-core-film-fabric-film,
film-fabric-film-inlay-film-fabric-film.
fabric-film-fabric-film.
fabric-core-fabric-film.
fabric-film-core-film-fabric-film.
fabric-film-inlay-film-fabric-film.
fabric-film-fabric-inlay-fabric-film-fabric-film.

In this list the layers may be made of a polymer optionally further comprising one or more metal components.

The design of the device as listed above may be combined with one or more other features as described herein, e.g. in the design one or more of the mentioned fabric film, core and/or lay may comprises metal in any of the compositions as mentioned elsewhere herein In case two similar layers are used in the device e.g. two layers of fabric, these two layers need not be of similar materials or have similar properties.

The mentioned layers need not be visible in the product ready to use. The layers may also be relevant only in respect of the polymeric components used, and one or more different metals may be distributed throughout or substantially throughout the device. Preferred is when the at least one of the outermost layers comprises less than 50% metal at the surface, such as less than 40%, e.g. less than 30%, such as less than 20%, such as less than 10%.

In the preferred embodiments described in the list above fabric comprises the first and/or third polymeric component as described elsewhere herein, and film, inlay and core comprise the second polymeric component as described elsewhere herein. The polymeric layers may each further comprise one or more metal components.

The layers of fabrics within a device may be similar or may be different in the construction. Also one or more layers of fabrics within a device may differ from the other layers of fabrics. Similar situations can be obtained regarding the film, inlay and core. Film, inlay and/or core of a single device may be different in construction.

By ‘different in construction’ is meant that the layers of interest can be produced by different materials or partly by different materials or the process of manufacture is different thus giving the layers different properties.

The constitutions of a product mentioned above may be surface coated by plasma polymerisation.

The polymeric material as described herein optionally further comprising one or more metal components can also be used to cover prostheses of other materials, such as standard prostheses.

In another aspect of the invention the medical device comprises one or more layers of fabrics which may be surface coated by plasma polymerisation.

Features of the Product

The thickness of the polymeric product is determined by the number of polymer layers optionally comprising one or more metal components and the dimension of these layers in accordance to the requirements of the medical device. The total thickness of the polymeric product is preferably between 0.001 and 40 cm thick, such as between 0.005 and 30 cm, preferable between 0.01 and 20 cm, more preferable between 0.02 and 10 cm, further preferable between 0.03 and 8 cm, yet further preferable between 0.04 and 5 cm, most preferable between 0.05 and 2 cm. In another embodiment the preferred thickness of a device is about 3 mm.

The surface area of a medical device may be between 1 cm and 200 Cm².

The surface dimension of a medical device comprising the polymeric layered structure as described herein may be between 0.01 to 40 cm according to length and width, such as between 0.05 to 35 cm, for example between 0.09 to 30 cm, preferable between 0.1 to 25 cm, more preferable between 0.2 to 23 cm, further preferable between 0.3 to 19 cm, yet further preferable between 0.4 to 17 cm, most preferable between 0.5 to 15 cm. Other preferred sizes of the surface dimension of a medical device may be between 0.5 to 8 cm according to length and width, such as between 0.5 to 7 cm, for example between 0.5 to 6 cm, preferable between 0.5 to 5 cm, more preferable between 0.5 to 4 cm, further preferable between 0.5 to 3 cm, yet her preferable between 0.5 to 2 cm, most preferable between 0.5 to 1 cm.

The surface dimension according to length and width of layers of fabric in a medical device as described herein may be substantially equal, equal or different from the surface dimension of the medical device. Preferred is a size where any surplus of fabric is removed following manufacture of the medical device.

To enclose one or more inlays with fabrics, the size of fabric according to the surface dimensions length and width may be the same as for the inlay, substantially the same as for the inlay or somewhat larger than the inlay. One or more inlays may be enclosed by two or more layers of fabric. The layers of fabric may have surface dimensions adjusted to cover all the inlays, although the inlays may have distance between each other. Two or more inlays of a device may or may not be positioned in between the same two layers of fabric.

The surface dimension according to length and width of layers of film in a medical device as described herein may be substantially equal, equal or different from the surface dimension of the medical device. Preferred is a size where any surplus of fabric is removed followed manufacture of the medical device.

The surface dimension according to length and width of layers of core in a medical device as described herein may be substantially equal, equal or different from the surface dimension of the medical device. Preferred is a size substantially equal to the surface dimension of the medical device.

The surface dimension according to length and width of layers of inlay in a medical device as described herein may be substantially equal, equal or different from the surface dimension of the medical device. Preferred is a size where the inlay is smaller than the surface dimension of the manufactured medical device.

To increase the strength of the polymeric product, the layers of fabric may be turned according to each other, hereby the fibres of the different layers of fabric is positioned into different directions. The fabric may be turned between about 0 to about 90 degree, such as between 10 and 80 degree, preferred is between 20 and 70 degree, more preferred is between 30 and 60 degree, further preferred is between 38 and 52 degree, yet further preferred is between 42 and 48 degree, most preferred is about 45 degree in relation to the former and/or next layer of fabric.

In an embodiment at least part of the polymeric component or polymeric components optionally comprising one or more metal components, of the device, is suitable for cells to grow into it. Preferred is when the surface of the device is in a condition suitable for cells to enter and grow into this surface. This in-growth of cells secure the device within the joint of the mammal.

The medical device may be secured with a collar and can be of any design and construction as mentioned elsewhere herein.

Method for Preparation

Polymers may be prepared by methods known to the person skilled in the art. Chemical catalysis, thermal induction or photo induction are anecdotal non-limiting examples of methods of preparing the polymers.

The polymeric product optionally Her comprising one or more metal components may be prepared according to the descriptions above by connecting stacked polymer layers by heating. The heating temperature is selected below the melting temperature of the fibre crystallites in order not to loose the crystallinity of the polyethylene fibres and to a level where the fibres of the fabrics are not melted, destroyed or damaged, ie. below 250 degree Celsius, and above the melting temperature of the polyethylene plastomer, ie. in the range between 80 and 250 degree Celsius, such as between 90 and 240 degree Celsius, preferable between 100 and 230 degree Celsius, more preferable between 110 and 220 degree Celsius, further preferable between 120 and 210 degree Celsius, yet her preferable between 130 and 200 degree Celsius, most preferable between 140 and 190 degree Celsius.

In an embodiment the heating temperature is preferably between 90 and 200 degree Celsius, such as between 95 and 195 degree Celsius, preferable between 100 and 190 degree Celsius, more preferable between 105 and 185 degree Celsius, further preferable between 110 and 180 degree Celsius, yet further preferable between 115 and 175 degree Celsius, most preferable between 120 and 170 degree Celsius.

In a further embodiment the heating temperature is preferably between 90 and 180 degree Celsius, such as between 95 and 170 degree Celsius, preferable between 100 and 160 degree Celsius, more preferable between 105 and 155 degree Celsius, further preferable between 110 and 150 degree Celsius, yet further preferable between 115 and 145 degree Celsius, most preferable between 120 and 140 degree Celsius

When heated the short chain polymers of the core or the film or the inlay penetrate into the fibres or filaments or strands of the fabrics, and hereby mechanically connect or bond the polymer layers to each other. In a preferred embodiment the temperature is selected to a level where the main part of the fabrics is not melted, but a thin layer constituting a low number of polymer chains or fibres of the outer part of the outermost fabrics of the polymeric product is melted. The heating process may be provided in vacuum and under pressure.

In a further preferred embodiment the temperature is selected to a level where the performances of the reinforcement fibres are not damaged.

The polymer product which may comprise stratified polymer layers as described above, and which has been subjected to the heating process, may be stored at room temperature until use. The polymeric product optionally comprising one or more metal components is preferable capable of being stored for long periods of time, such as several years. Storing is performed in dry conditions at room temperature and in darkness or at least without direct sunshine to the product. Dry conditions may be humidity of about 10-90%.

The medical device prepared by the described method can be of any design and construction as mentioned elsewhere herein.

Shaping the Medical Device

In the heating process described previously or following a storage period of the fused polymer layers, the polymeric product comprising the polymer layers may be subjected to vacuum and may simultaneously be pushed or pressed into a mould to form the polymeric product. The vacuum process prevents the formation of bubbles and protects the polymer from oxidative degradation. Shaping can also be performed without vacuum but due to pressure optionally combined with heating the polymeric product.

The vacuum of the method described above may be below 500 mbar, preferable below 300 mbar, more preferable below 100 mbar, further preferable below 50 mbar, yet further preferable below 10 mbar, most preferable below 1 mbar.

To secure the desired shape of a device, the pressure of the device in the shaping process may be maintained until the polymer product is cooled preferable to room temperature. This cooling under pressure secures consolidation of the product.

The pressure in the process described above is a pressure high enough to press the product into a mould, the pressure may be a low pressure performed for a long period or a high pressure performed for a short period, or a pressure in between. Low pressure in this context is the pressure just enough to press the product into a mould.

The polymer product optionally comprising one or more metal components pressed into a shape as described above, may be stored at room temperature for long periods of time, such as several years.

In an embodiment the polymeric product optionally comprising one or more metal components may be produced with one or more apertures, holes, gaps, perforations or hollows. The apertures etc. may constitute an improved attachment and/or optimise the function of the device. The improved attachment may be obtained without further processing as the apertures may constitute a shape of the device in a way that the device better fits into the location of the body. The apertures can also be utilised to fasten the device within the body.

Fastening methods are known to persons skilled in the art, and are hereby incorporated.

The apertures etc. may be created simultaneously with the shaping of the polymeric product, hereby the mould has points, tips or peaks, which create the apertures in the polymeric product. Another method of producing apertures etc. is to make a hole by a drill or another boring, cutting or pressing apparatus. Followed the formation of apertures etc. by drilling, cutting or pressing holes, the edge of the apertures on the polymeric device may be closed.

To the polymeric material optionally comprising one or more metal components may be attached a component, the component being polymeric or non-polymeric. The attachment may constitute part of a prosthesis or provide an anchorage point.

The medical device of the shape described can have of any design and construction and be of any material as mentioned elsewhere herein.

Composition of Devices

The device may be layered only with polymer layers optionally comprising one or more metal components, or a combination of metal layers together with polymer layers optionally comprising one or more metal components.

In an embodiment the number of layers of polymer optionally comprising one or more met components may be similar to or lower than the number of layers of metal. The composition may be according to the following list, where polymer means a polymer layer optionally comprising one or more metal components, The list is not exhaustive:
a metal-polymer
metal-polymer-metal
metal-polymer-polymer-metal
metal-polymer-metal-polymer-metal
metal-polymer-metal-polymer-polymer-metal
metal-metal
metal-metal-polymer
metal-metal-polymer-polymer
metal-metal-polymer-metal
metal-metal-polymer-polymer-metal
metal-metal-polymer-metal-metal
metal-metal-metal

The list does not indicate the orientation in respect of outer and inner or upper and lower layer of the device. In a device with two or more polymer layers, these layers can be of a similar composition or be different in composition. In a device with two or more metal layers, these layers can be of a similar composition or be different in composition.

In a device which comprises one or more layers of metal located next to each other the metal layers may be only soft metals, only hard metals or a combination of soft and hard metals.

In an embodiment the number of layers of polymer optionally comprising one or more metal components may be similar to or higher than the number of layers of metal. The composition may be according to the following list, where polymer means a polymer layer optionally comprising one or more metal components, The list is not exhaustive:
polymer-polymer
polymer-metal
polymer-metal-polymer
polymer-metal-metal-polymer
polymer-polymer-metal-polymer
polymer-polymer-metal-polymer-polymer
polymer-metal-polymer-metal-polymer
polymer-polymer-polymer

Devices produced from layered structures may be any devices as mentioned elsewhere herein.

In an aspect of the invention the prosthetic device is not a layered product, or not produced from layers of polymer and/or metal. The device may be produced from on single polymer product optionally further comprising one or more metal components or may be produced entirely or substantially entirely from one type of metal or metal alloy.

In a device with a non-layered structure different areas of the device may be made from two or more polymer.

In an embodiment the device is made of a polymer which is suitable of injection moulding, to this polymer is added one or more types of metal fibre and/or metal powder. Optionally also polymer fibre can be added. Preferably the polymer which is suitable of injection moulding is one or more polyolefin. More preferred the polymer is polyethylene and/or polypropylene. Preferably the metal is selected from titanium, gold, silver and/or chromium/cobaltum.

In an embodiment the device is produced by shaping a polymeric material comprising fibre and/or powder of metal, optionally polymer fibre may also be added to the polymer. The shaping may be performed by injection moulding.

In an embodiment a device, e.g. an acetabular cup or an interpositional arthroplasty can be made of a polymer which is suitable of injection moulding, to this polymer is added one or more types of metal fibre and/or metal powder. Optionally also polymer fibre can be added. Preferably the polymer which is suitable of injection moulding is one or more polyolefin. More preferred the polymer is polyethylene and/or polypropylene. Preferably the metal is selected from titanium, gold, silver and/or chromium/cobaltum.

In another embodiment a device, e.g. an acetabular cup or an interpositional arthroplasty can be made of a 3D network or a mat of metal fibre and/or polymer fibre. The mat is filled with a polymer with a lower melting point than the polymer fibre and the device is subjected to heat and pressure to produce a device comprising fibre of metal and/or polymer fibre in a matrix of polymer.

The 3D-network may have knitted, crochet and/or weaved structure, or a structure described elsewhere herein.

The metal to be used in the 3D-network may be any suitable metal. Preferred is a metal selected from titanium, gold, silver and/or chromium/cobaltum.

The polymer fibre to be used in the 3D-network may be fibre of any polymer described elsewhere herein. Preferred is a polymer fibre of polyethylene or polypropylene. More preferred is UHMWPE.

Devices produced without a layered internal structure or without laying layers towards each other in the production process may be any devices as mentioned elsewhere herein.

Collar

Following the shaping of the polymeric product optionally comprising one or more metal components as described elsewhere herein, any surplus of polymeric material can be removed e.g. by cutting off. Cutting off the surplus of polymeric material leaves a polymeric product with right angle edges. These edges have to be rounded to secure no damage of the product is performed within the animal or human body when function as a medical device within the body.

In an embodiment the rim of the device may be treated to fix loose ends of fibre or strands. The rim may be closed by sewing or by fastening a polymer ring or a mead ring. When using a ring to close the rim of the device, the ring may be 0.5-5 mm thick, preferred is 3 mm.

In another embodiment a collar is placed on the medical device when surplus of material is removed. The collar can be moulded directly on the device e.g. by injection moulding.

In another embodiment a collar is moulded before positioned onto the edge or edges of the device. Preferred is when the collar is injection moulded. The collar can be secured to the device by ultrasound welding, gluing, sewing and/or laser welding.

The collar as described above can be of any material mentioned in the description of the first, second or third polymeric component optionally comprising one or more metal components. The collar material of the medical device can be the same material or a different material as actually used for the first, second or third polymeric component optionally comprising one or more metal components. Preferred is when the collar includes fibre e.g. of UHWMPE or LDPE. More preferred is collar of LDPE. Preferred is also when the collar includes nano-structured composite of polymer and/or metal. Further preferred is when the collar includes short and long composite of polymer and/or metal. Most preferred is when the collar is produced of the same polymeric component optionally comprising one or more metal components as actually used for the core or film, due to compatibility between the materials. Within the process the polymeric component of the collar may melt together with the polymeric component of the film and/or core. Also preferred is when the collar material includes nano-fibre or short fibre of polymers and/or metals.

In an embodiment a cup shaped medical device where the hat brim of surplus of material is removed a pre-moulded collar of LDPE optionally comprising one or more metal components is attached on the cut edges.

The collar closes the edge, gives the cup strength, and includes or support the marker e.g. in the form of a gold thread. Furthermore the collar modify the friction.

Instead of mounting a collar on the edge of the device, it is possible to bead or flange the edge or edges. This treatment can be performed under heat.

A collar that is pre-moulded before mounted to the edges of a medical device is easier to handle, and has economical benefits compared to injection moulding a collar onto the edges.

It can be difficult to mould a collar directly on the medical device, as the device may become soft at the temperature and pressure at which the collar is injection moulded onto the edge of the device. Hereby there is a risk of deformation of the outer area of the device. With a pre-moulded collar that is secured to the edge by ultrasound welding, gluing, sewing and/or laser welding the edge and outer area of the device is not at risk of the mentioned deformation.

The methods of fastening the pre-moulded collar to the medical device secure a safer attachment between the collar and the edge and the outermost part of the outer area of the device. The material of the collar may adhere to both the first, second and/or third polymeric material of the device, these polymeric materials optionally further comprise one or more metal components. Preferred is ultrasound welding to attaching the pre-moulded collar to the medical device.

The pre-moulded collar has a dimension that secure that the collar when attached to the medical device covers at least 0.5 mm of the outer area of the upper surface when measured from the edge. A similar dimension may be covered at the lower surface of the device. The collar need not cover equal dimensions of the upper and lower surface.

In an embodiment the pre-moulded collar has a groove wherein the marker can be placed. Hereby the marker may be located at the edge of the device and the collar encloses or support the marker Preferred is a thread or wire of metal.

The marker can be in the form of one single unit or in the form of at least two units, and where the at least two units are placed non-homogeneous within the device. Hereby the rotation of the device within the joint ban be detected as described elsewhere herein.

In an embodiment the collar includes and/or supports at least one marker. These markers are described herein below. By including these markers in the collar there is no need of placing marking particles In holes of the device. Thus the incorporating of a marker in or supported by the collar eliminates the step of drilling in the device.

In a preferred embodiment a cup-shaped or approximately cup-shaped medical device has a top and a skirt with a thickness larger than the thickness of the top, and where the edge of the skirt is enclosed by a pre-moulded collar. The collar can include a marker as described elsewhere herein.

Collars as described herein may be use for a medical device of any design and construction as mentioned elsewhere herein.

Markers

In an embodiment markers are placed within the medical device. The markers can be used to visualise, trace or in other ways show the position of the medical device when inserted into a body. Visualisation can be performed by methods known to a person skilled in the art which is hereby incorporated. One method is X-ray identification. The material of the markers can be any material, which can be placed within the polymeric material optionally comprising one or more metal components and can be detected from outside of the body.

In one embodiment the markers are contrast balls. Before closing the rim with a method described elsewhere, contrast balls are placed within the device. The contrast balls which can be any suitable colour such as but not limited to blue, red or green, can be placed in small holes drilled in the device. The drilled holes can be made at a right angle to the surface established when surplus of material is removed. Fastening a ring to the device or moulding a collar as mentioned above closes the holes. The number of contrast balls are optional, in a cup device 3-10 contrast balls may be utilised, 7 contrast balls is preferred.

In another embodiment the marks are made of metal. The shape of the markers is optional. Preferred are balls of metal. More preferred are markers of stainless steel or tantalum. Most preferred are balls of stainless steel or tantalum. The markers are placed within holes of the medical device drilled from the cut edge appearing when surplus of material is removed. The number of markers is optional. The number of markers is preferably 1-10, such as 2, e.g. 3, such as 4, e.g. 5, such as 6, e.g. 7, such as 8, e.g. 9, such as 10. The placing of the markers may be optional. Preferably the markers are placed asymmetrically around the cut edge of the medical device. This asymmetric placement ensures the possibility to measure if the medical device changes position when implanted in the body. In a cup-shaped medical device the asymmetric placement of the markers is preferable an asymmetric placement according to the circle comprising the cut edge of the medical device, hereby it can be visualised within the body whether the cup rotates. The cut edge is closed with a method described elsewhere herein.

In a fisher embodiment the markers are small pieces of the marking material. The small pieces are placed within the core or inlay when these are moulded.

In another embodiment the markers are formed as threads or wires, and are placed within the core, inlay, film or fabric when these are produced. Threads of markers can also be placed between the fabric and any of the core, inlay or film when the medical device is constructed from the components.

The threads or wires may be of any material that is possible to detect from the outside of the body of a mammal. The material may be metal, barium sulphate, Teflon, master batch, dye, contrast medium, or filler in the collar for detecting the implant and the placement of the implant Examples of metal to be used as marker may be gold tantalum and titanium, which can be used individually or jointly.

Preferred is when the marker is a metal thread or wire, and the marker is non-homogenous placed in the cup, hereby it is possible to detect rotation of the implant within the joint of the mammal.

Markers as described herein may be use for a medical device of any design and construction as mentioned elsewhere herein.

The medical device with the marker may be detected by any suitable detection method, such as X-ray, magnetic resonance scanning, CT scanning, MRI scanning, or PET.

Preferred is when the first, second or third polymeric component optionally comprising one or more metal components or another component of the collar is a X-ray sensitive component.

The marker is preferable not a magnetic marker when MRI scanning, although a little magnetic character of the marker and/or device is acceptable.

The marker can be a magnetic marker when X-ray detecting is to be used. For X-ray detection the marker may include heavy atoms, and/or gold octant.

In PET detection, electronic charged positrons are detected, this result in inverted contrast when liquid is injected into the joint.

Fluorine NMR can be used to detect the device with Teflon threads or Teflon wires, where the frequency of fluorine is utilised. In Fluorine NMR it is only possible to observe the thread with fluorine. The thread may include e.g. cilice, carbon, selenium.

Smoothness of the Surface

The smoothness of the surface of the device is important as this has a connection with the level of pain as well as a connection with pain relief of the mammal in whom a medical device is located within a joint.

In an aspect of the invention the medical device comprising at least a first and a second side, wherein the at least first side is made of a first polymeric component and wherein the first and/or second side has a frictional resistance of less than 1 Newton. Preferred is less than 0.5 Newton. More preferred is less than 0.2 Newton.

The device becomes more smooth when located in a joint and this joint is in function. The surface of the device will be exposed to some wear, hereby the surface becomes more smooth. By producing a device which when implanted is more smooth than normal, pain relief of the individual wherein the device is implanted is obtained faster.

In an embodiment the first polymeric component has a frictional resistance of less than 0.5 Newton and the first polymeric component is located in at least a first area where the device is subjected to wear when the device has been implanted into an individual.

In an embodiment the wear in the at least first area is due to friction made by a bone and/or a medical device.

In a joint in movement, the areas of the device which is always subjected to contact with a bone or another part of an implant is subjected to most wear.

In an embodiment the second side is made of a first and/or a second polymeric component. The polymeric component of this second side may have a similar or another smoothness as the component of the first side.

The surface of the device can be made more smooth by moulding pressing the device an extra the in succession to the preparation of the device. Another method to improve the smoothness is to rub the device for a duration which is suitable to obtain a desired smoothness.

In an embodiment the first and/or second side further includes at least one cavity. The at least one cavity may have a diameter of at least 0.05 mm when measured at the outside of the first side.

The cavities of the device provide more space for liquids such as synovial fluid, physiological salt solution or another biocompatible liquid. Hereby is the friction coefficient lowered, the wear is lowered, a lesser degree of pain is obtained by the mammal wearing the device and the durability of the device is increased.

In an embodiment the at least one cavity has a depth of at least 0.01 mm.

The cavities may be in the form of grooves. These grooves may be in straight lines or in patterns e.g. waving lines or zigzag lines.

In one embodiment a device has a smooth surface together with cavities. Especially the surface is smooth between the cavities in the areas which is subjected to wear in the joint. Preferred is a cup-shaped device with a smooth surface and cavities.

The medical device with the smoothness and cavities as described can have of any design and construction and be of any material as mentioned elsewhere herein.

Perforations

Medical devices with perforations or apertures provokes an increased flow of liquids such as synovial fluid within the joint.

In an aspect of the Invention a medical device comprising at least a first polymeric component, wherein the device has at least one through-going perforation which is not for ligaments.

In an embodiment the at least one perforation has a diameter of at least 0.01 mm.

In an embodiment the at least one perforation is located in at least one area where the device is subjected to wear.

In an embodiment the perforations are located homogeneous over substantially the entire device.

In an embodiment the perforations can conduct liquid from a first side of the device to a second side of the device and/or from the second side to the first side. Hereby the liquid can be displaced within the joint in respect of the movement of the joint.

The liquid may be synovial fluid or physiological salt solution or another biocompatible liquid.

In an embodiment the device further comprises an inner volume between the first and second side and inside this inner volume the liquid can be within.

This volume may be an empty volume when the device is inserted into the joint. Liquid within the joint can enter this volume. The volume can also include liquid when inserted into the joint or be filled after it is inserted. Hereby the inner volume comprises a storage of liquid.

The inner volume can comprise a network of at least one polymeric component optionally comprising one or more metal components. This polymeric component can be polymeric fibres and if present the metal components can be metal fibre, where the empty space constitute space for liquid as described above.

The medical device with the perforation and/or inner volume as described can have any design and construction and be of any material as mentioned elsewhere herein.

Self-Healing Characteristics

In an aspect of the invention a medical device comprises at least a first polymeric component optionally comprising one or more metal components, wherein the polymeric component is self-healing when subjected to injury before implantation, during implantation and/or after implantation.

In an embodiment the polymeric component is a composite polymeric component optionally comprising one or more metal components.

The device includes a self-healing polymer systems to auto-repair cracks formed in plastics or composites materials. Microencapsulated monomers healing agent) and initiators are incorporated into a polymer matrix to produce a polymer composite capable of self-healing. Cracks formed in the material ruptures embedded micro-capsules releasing healing agent into the cracks. Polymerisation of the healing agent is triggered by contact with an embedded catalyst, repairing the cracks. Alternatively the polymers comprising the device can be selected from chemical structure bearing self healing properties by being able to revert the chemical changes occurring upon rupture

In an embodiment the healing agent is monomers of the first polymeric component and/or of a second polymeric component.

The medical device with the self-healing characteristics as described can have any design and construction and be of any material as mentioned elsewhere herein.

Two Congruent Cups

In another aspect of the invention a medical device comprises at least a first unit with at least a convex surface and a second unit with at least a concave surface, where the convex and concave surface is congruent with each other and the first unit fit partially or entirely into the second unit, and wherein the first and/or second unit comprises at least one polymeric component optionally comprising one or more metal components.

In an embodiment first and second unit have substantially similar size.

In an embodiment the first and second unit have different sizes.

In an embodiment the first and second units are connected or substantially connected by the convex and concave surface. Hereby the one unit fits into the other unit. The concave surface can be at least 0.01 mm smaller than the convex surface.

In an embodiment the units are movable compared to each other, and the units continue to be in contact during a movement and/or return to be in contact when a movement is finished. The first unit and/or the second unit may be attached to an implant and/or a bone.

In an embodiment the it unit and/or the second unit can function without being attached to an implant and/or a bone.

In one embodiment the two units are two cup-shaped units, which are secured or attached to a bone or implant or located at a bone or implant or within a cavity. Between these cups may be located gas in the form of air liquid such as synovial fluid or a physiological salt solution or another biocompatible liquid.

The medical device with the two units, one with a convex surface and the other with a concave surface as described can have any design and construction and be of any material as mentioned elsewhere herein.

A Fabric to be Folded to Constitute Two Polymeric Layers

In an aspect o the invention a medical device comprises at least an upper layer, a first middle layer and a lower layer, wherein the upper layer and the lower layer is made from one uninterrupted piece of at least a first polymeric component optionally comprising one or more metal components.

In an embodiment the piece of at least a first polymeric component optionally comprising one or more metal components is in the form of a fabric. The piece of at least a first polymeric component optionally comprising one or more metal components is constructed in one piece, which is folded around an axis and hereby forming the upper and lower layer.

In an embodiment the upper and lower layer are similar or substantially similar in size. The upper and lower layer can also be of different sizes.

In an embodiment the at least first polymeric component is in the form of fibres.

In an embodiment the piece of at least a first polymeric component is in the form of a tube or pipe before folded around the axis. The tube may have a uniform diameter along the tube. Furthermore the tube may contract in a first and/or a second end of the tube. The tube may also contract in the axis.

In an embodiment the piece of a first polymeric material optionally comprising one or more metal components further comprises at least one aperture. This at least one aperture may be located at the axis or in an area that is subjected to a restricted amount of wear. The number of apertures is preferably between 1 and 20.

When the piece of a first polymeric material optionally comprising one or more metal components is folded around the axis, the first middle layer comprises empty space. This space may be filled with a second polymeric component optionally comprising one or more metal components. One or more of the apertures can be utilised to fill the device with a polymeric component optionally comprising one or more metal components.

To the device a collar as described elsewhere herein may be attached. The collar may include or support a marker as described elsewhere herein. The marker can be a gold thread.

In an embodiment the fabric and hereby the tube is made by weaving, knitting or crocheting.

In an embodiment the axis is located substantially at the middle of the tube and the axis is parallel or substantial parallel to at least one opening of the first and second end of the tube.

The first middle layer can be of a second polymeric component optionally comprising one or more metal components which may be a component described in respect of film, core or inlay.

The medical device with a fabric to be folded to constitute two polymeric layers as described can have any design and construction and be of any material as mentioned elsewhere herein.

Bag with a Firm Shape

In an aspect of the invention a medical device comprises a bag of a first polymeric component optionally comprising one or more metal components, wherein the bag has a firm shape in at least one dimension.

In an embodiment the bag has a firm shape in at least two dimensions. Furthermore the bag may have a firm shape in at least three dimensions.

In an embodiment the first polymeric component optionally comprising one or more metal components encircles an inner volume, the inner volume may be filled with a second polymeric component optionally comprising one or more metal components before implantation, during implantation and/or after implantation. The second polymeric component optionally comprising one or more metal components may be a component described in respect of film, core or inlay.

In an embodiment the second polymeric component comprises, fluid polymeric component at least when the polymeric component is filled into the bag and/or particles of the polymeric component, the particles being spherical, lentil-shaped, egg-shaped, pyramid-shaped, and/or star-shaped. The second polymeric component may comprise one or more metal components

In an embodiment the first polymeric component optionally comprising one or more metal components is a fabric and the fabric is made e.g. by weaving, knitting and/or crocheting.

The medical device may be manufactured individually according to the dimensions measured in a joint of a mammal, where the device is to be inserted.

Medical devices which are soft at the time of implantation may be inserted into the joint of a mammal by arthroscopy. The device can continue to be soft within the jog or harden. The device can also be filled with another component as described elsewhere herein.

The medical device with a bag with a firm shape can have any design and construction and be of any material as mentioned elsewhere herein.

Strengthening Implants with Fabric

In an aspect of the invention a medical device comprises at least a first surface area, wherein at least a first polymeric component is attached to the first surface area. The device may at least in the first area be subjected to wear when the device is located in a mammal individual.

In an embodiment the device is made of polymer, bone and/or metal. The first polymeric component may constitute at least 1% of the surface of the device.

In an embodiment the first polymeric component optionally comprising one or more metal components may be located at the outside of the device in areas that is subjected to wear. The first polymeric component optionally comprising one or more metal components can be in the form of a fabric. The fabric can be made by weaving, knitting and/or crocheting.

One or more layers of fabric can be attached to the first surface area. These layers of fabric can be connected by a second polymeric material optionally comprising one or more metal components e.g. in the form of a film or core as described elsewhere herein. The fabric may be suitable for in-growth of cells

In all embodiment the at least first polymeric component further is placed onto the device in areas that is subjected to a less amount of wear and/or is not subjected to wear.

Preferred is a hip-joint prosthetic device in which the cup is fabricated from a multiplicity of layers of fabric. The number of layers can be between 2 and 100, preferred is between 3 and 50, also preferred is between 4 and 25, more preferred is between 5 and 15. The layers of fabric may be connected by film or core as mentioned elsewhere herein. The layers of fabric may be of different structure in respect of fibre thickness and e.g. weaving-method, and may also be made of different polymeric materials. The cup may be manufactured in a composite structure, or individually layers of fabric may have a composite structure.

The medical device which is strengthened with a fabric and the fabric used to strengthened the device can have any design and construction and be of any material as mentioned elsewhere herein.

Finishing Treatment of a Medical Device

The present invention in particular relates to material formulations intended to meet the specifications of durability, bio-compatibility, and strength. These properties are obtainable by treating polymer materials, such as polyethylene, polypropylene or polyvinylpyrrolidone or combinations and co-polymers thereof as well as precursor materials for polymerisation, with high-energy electrons, gamma rays, photons, microwaves, ion implantation, plasma treatment, annealing, thermal radiation or another radiation to obtain ideal durability and bio-compatibility of the new, modified material. Treatment of the above-mentioned materials with radiation leads to cross-linking of polymers and thereby generating new, modified materials. Preferably, the polymer material is a cross-linked polyethylene or polypropylene material. More preferably the polymer material is a cross-linked polyethylene material.

The properties of the materials to be obtained by the cross-linking process are preferably resistant to tear and wear; and have good compressibility.

The medical device may be packed in a pouch, which is suitable for irradiation. Preferred are pouches of aluminium, more preferred are laminated pouches of PE, aluminium and PET, where PE (polyethylene) comprises the inner of the pouches and PET polyethylene-terephthalate) comprises the outer of the pouches.

A pouch with a medical device may be filled with nitrogen before it is made airtight. The medical devices are then subjected to irradiation to cross-link the polymeric material and sterilise the medical device.

In order to increase stability of the medical polymeric device the polymers of the shaped polymer product may be subjected to further treatment such as cross linking. In a preferred embodiment the cross lining treatment is conducted in order to cross link only a fraction of cross linkable polymers in the product. Accordingly, the products may be cross-linked by radiation, the cross-linking of the polymers may also be done by other methods known to the person skilled in the art. The radiation may be but is not limited to hi-energy electrons, gamma rays, photons, and microwaves. Cross binding the polymers improve the strength of the product. A preferred radiation process is cross-linking of fibres using treatment with accelerated electrons. As the cross-linking process takes place in the amorphous polyethylene regions, the optimal dose will depend on the Zion of amorphous polyethylene in the final device. The optimal radiation dose is preferably close to the gelation dose of polyethylene and thus lie between 10-10,000 kGy (0.1 and 100 Mrad), preferred is between 10 and 300 kGy, most preferred is 200 kGy.

The radiation can be performed in one uninterrupted treatment, where the complete dose of radiation is given to the material. The radiation process may also be performed in pulsing or interrupting treatments, where the total dose of radiation is given in 2-15 shorter with an interval of 1 to 60 minute. Preferred is 25 kGy given eight times with 10 min interruption between each radiation treatment (total 200 kGy). More preferred are two times 25 kGy Interrupted by 1 to 60 minutes and repeated 4 times with 10 hours to 1 day of interruption.

The radiation may be performed for the entire product or device or only part of the product or device is radiated by using a shield or screen between the irradiation source.

When using radiation, the radiation process described may be followed by annealing. The purpose of annealing is to eliminate long living fee radicals by a heat treatment of 80° C. for about 1-12 hours in vacuum. More preferred is 70-85° C. for about 16-24 hours in an inert atmosphere. Preferred is when the inert atmosphere is Nitrogen.

Typically, a device is prepared by a process comprising the following steps:

The device is formed under vacuum by pressing the laminated polymers optionally comprising one or more metal components in a mould of specified dimensions. The polymer optionally comprising one or more metal components is chosen from the above mentioned polymers.

After hardening the material as formed, or after swelling in a suitable solvent, the device may be subjected to high-energy electrons, gamma rays or another radiation in order to create cross-linking which will modify the mechanical properties of the material to meet the preferred specifications.

Finally, after removal of the swelling solvent the surface of the material may be treated to achieve good surface properties as described elsewhere.

The medical devices may be subjected to annealing when they are irradiated. Annealing is performed in an oven at about 80° C. for a few hours to remove residual free radicals. Or annealing is performed as described elsewhere herein.

Surface Coating

The surface of the device can subsequently be treated to modify surface properties such as wetting ability and/or biocompatibility. This surface treatment can be performed by plasma treatment, chemical grafting or by a combination of plasma polymerisation and chemical grafting. The material contacting with the biological surfaces may be smooth, biocompatible, preferably self-lubricating, and it should be wear-resistant so that particles generated due to wear are avoided in that his could otherwise result in foreign body reactions and cause further trouble to the function of the part of organism where the medical device is located.

Furthermore, the surface material should preferably be a material or a combination of materials having self-repairing properties so that fissures, cracks or other ruptures on the surface do not exceed uncontrollable levels. However, the surface material is preferably continuous with the material of the rest of the device, e.g. the material may gradually merge into the material of the fabric, film or core of the device, in this context continuous means that the surface material cannot be pulled away from the material beneath.

The surface of the material may be chemically treated so as to soften, rigidify or lubricate the surface of the device or parts thereof. The surface of the material may be coated so that the coating confers these properties, or may be treated so as to chemically alter the surface of the device so as to confer any of these properties. Alternatively, certain polymer surfaces may be modified by means of thermal or photolytic energy.

Without being bound by theory it is also believed that a wetted surface reduces the risk of having the immune system recognising the device when implanted, which would otherwise lead to adverse effects of the device.

In one embodiment the surface of the device may be coated by a plasma polymerisation, using low-power plasma equipment. The monomers used for the plasma polymerisation are any monomer forming a hydrophilic polymer by plasma polymerisation. Preferred are monomers forming polyvinylpyrrolidone and polyethylene-glycol like polymers, most preferred is 1-vinyl-2-pyrrolidinone.

The surface coating performed as described above has a thickness of 1 to 700 nm, such as between 10 and 500 nm, preferable between 20 and 400 nm, more preferable between 30 and 300 nm, further preferable between 40 and 200 nm, yet further preferable between 50 and 100 nm, most preferable between 60 and 90 nm.

In another embodiment the surface coating performed as described above has a thickness of 1 nm to 5,000 nm, such as between 5 and 2,500 nm, preferable between 10 and 1000 nm, more preferable between 30 and 500 nm, further preferable between 40 and 400 nm, yet further preferable between 45 and 300 nm, most preferable between 50 and 250 nm.

Plasma is ionised gas. In an artificial plasma to be used for plasma treatment and plasma polymerisation, the concentration of ionised species is preferably 0.1-10 ppm. Two phases exists in artificial plasma: A gas-phase comprising an energy corresponding to the surrounding temperature, usually room temperature. In a plasma-phase ions and electrons have an energy at approximately 2-10 eV.

The artificial plasma may be established by exposing a gas with electric field. The pressure of the gas is preferably 0.01-1 mbar. The electric voltage utilised is dependent of different features such as the pressure, the composition of the gas, electrode configuration, the size of the polymerisation chamber, and frequencies of the electricity. The voltage is typically 200-10,000V.

In a preferred embodiment of the plasma polymerisation 1-vinyl-2-pyrrolidinone (VP) may be polymerised to polyvinylpyrrolidone (PVP) in a plasma with low energy. The plasma functions as an initiator for the polymerisation by formation of radicals in the surface of the element to be coated. From the radicals the polymerisation process takes place where monomers of VP polymerise to PVP. A low energy is necessary not to destroy the monomer VP in the gas-phase as well as the polymerised PVP. In a preferred embodiment the energy is 0.1-1 W/L

In the plasma polymerisation treatment a carrier gas is used, preferred is an inert gas, such as argon or helium.

The chamber for performing the plasma treatment is constructed to perform a homogeneous surface coating of the device by the plasma polymerisation process.

The surface coated polymeric product is preferably sterilised by radiation or by heating. The radiation can be but is not limited to high-energy electrons, gamma rays, photons, microwaves.

The polymeric product may by crosslinked and sterilised simultaneously by treating with ionizing radiation or by heating. Preferred is cross-linking by radiation.

Mechanical Properties

The structure of the material of a device may comprise a layered or laminated structure, a core of one material or one or more interposed layers with different properties enabling an overall function of the devise suitable for providing a spacer function and/or to exert pressure distribution of joints and/or to provide at least part of the sliding/rotating movement of joints by internal movement of the device, or relevant part of the device. However, it is preferred that the material itself does not comprise interposed layers resulting in sliding between the layers and thereby tear on the mating surfaces within the device. Accordingly, the body of the device should be one continuous solid or semi-solid material.

Mechanical properties for certain relevant polymers are described by Szycher (Szy-cher, M. (editor), sponsored by SPE, Society of Plastics Engineers, Inc. Biocompatible Polymers, Metals, and Composites, pp. 725-727, 757-61).

Mechanical properties of polymers are controlled by the elastic parameters, the three moduli: elastic, shear, and compressive moduli. These parameters are theoretically interrelated. A modulus is the ratio between the applied stress and the corresponding deformation. The reciprocals of the moduli are called compliances. The three elastic moduli have the dimension: force per unit area, (N/m² or Pa). Polymers are not normally ideal elastic bodies, but under load they show (time dependant) viscoelastic properties. By taking the load into consideration, the properties should be viewed according to this dilemma. Also, ideal elastic properties and ultimate properties are influenced by the viscoelastic properties.

Ultimate tensile strength is a measure of the stress required to cause the material to rapture in tension. Ultimate elongation is the percent stretch of the material before it ruptures in tension. Elongation (%) is measured as

$$Elongation ( percent ) = \frac{{S}_{B} - {S}_{o}}{{S}_{o}} \times 100$$

where S_{B}=observed distance between bench marks of the stretched specimen at rupture, and S₀=the original distance between bench marks.

TABLE 1
Elastic parameters and their definitions

| Elementary mode of | | |
|---|---|---|
| deformation | Elastic parameter | Symbol |
| Isotropic | Bulk modulus | K |
| (hydrostatic) | bulk compliance or | κ |
| compression | compressibility | (κ = 1/K) |
| Simple shear | Shear modulus or rigidity | G |
| | Shear compliance | J(J = 1/G) |
| Uniaxial extension | Tensile modulus or | E |
| | Young's modulus | |
| | Tensile compliance | S(S = 1/E) |
| Any | Poisson ratio | ν |

Examples of ranges of the mechanical properties of the device are mentioned below. However, it should be contemplated that not all of the following characteristics may be fulfilled by the material of the prosthetic device since, as explained above, the numerous properties of the material are theoretically interrelated. Accordingly, conflict in fulfilling all parameters within the stated ranges may occur. In one embodiment, the

| Symbol | Definition |
|---|---|
| K | $\frac{Hydrostatic Pressure}{Volume change per unit volume} = \frac{p}{ΔV / {V}_{0}} = \frac{{pV}_{0}}{ΔV}$ |
| κ (κ = 1/K) | reciprocal of foregoing |
| G | $\frac{Shear force per unit area}{\begin{matrix}Shear per unit distance \\ between shearing surfaces\end{matrix}} = \frac{F / A}{tan γ} = \frac{τ}{tan γ} = \frac{τ}{γ}$ |
| J (J = 1/G) | reciprocal of foregoing |
| E | $\frac{\begin{matrix}Force per unit \\ cross \text{-} sectional area\end{matrix}}{Strain per length} \frac{F / A}{ln ( L / {L}_{0} )} = \frac{σ}{ɛ} = \frac{F / A}{ΔL / {L}_{0}}$ |
| S (S = 1/E) | reciprocal of foregoing (strain/stress) |
| ν | $\frac{Change in width per unit width}{Change in length per unit length} = \frac{lateral contraction}{axis strain}$ |

prosthetic device according to the invention is a device wherein the material of the device or at least the part of the device which exerts the pressure distribution and/or the part which exerts the sliding/rotating movement in the joint when the joint is loaded has/have one or more of the following properties (under biological conditions (37° C., physiological salinity)): A compressive modulus (K) of at least 2000 MPa, a shear modulus (G) of at least 1 MPa and an elastic module (E) of at least 10 MPa.

Furthermore, certain requirements to the material under stress with forces that ultimately leads to disintegration can be expressed. Based on the elasticity parameters for the material, the properties of the material with respect to pressure, elongation, torsion and displacement in the range where the material responds elastic can be estimated. The ultimate limits should preferably be within ±20% of the range of elastic response. As a consequence thereof the limits for the ultimate properties (ultimate compression strength, tensile strength, torsional strength, shearing strength) can be derived. Furthermore, the material should have an “ultimate percentage elongation” of at least 20%.

The materials according to the invention may be a “quasi elastic” material. Y. Shikinami and H. Kawarada, Biomaterials 19, 1998, pp. 617-635, discuss that many materials of biological origin, has a J-form in a stress vs. strain curve, whereas may synthetic materials has an S-form.

Preferably, the critical surface tension (y_{c}) values should be within the “zone of bio-compatibility” corresponding to the range of about 20-30 dynes/cm (as defined by Lelah M. D., Cooper, S. L., Polyurethanes in Medicine—CRC Press, Inc. Boca Raton, Fla., pp. 59-62 and 92-93).

Additives

A device constructed from the polymeric product optionally comprising one or more metal components may comprise biologically active additives. Medication or biological active substances can be used as additive to the device to facilitate healing, minimise destruction or with other therapeutic goals, such as pain relief, anti-inflammation, oncology treatments, stimulation of bone growth and/or anti-infectious agents. Also, biological osteogenic or chondrogenic, chondral inductive, and/or chondral conductive materials may be added to the device. In particular patients suffering from osteoporosis or other bone degenerating conditions may benefit from having devices comprising osteogenic inductive materials implanted.

The medication or biological active substances can be used as additive to the device to facilitate cell growth, such as osteocytes, osteoblasts, chondrocytes, chondroblasts, mesenchymal cells. Cartilage inducing factor may for example be the factors described in U.S. Pat. No. 4,774,322 and U.S. Pat. No. 4,843,063.

In another preferred embodiment, additives such as lubricants, dyes, stabilizers and other process enhancing compounds are incorporated into the polymeric mixture. Such compounds may not necessarily enhance the strength or structural integrity of the final polymeric matrix, but do aid in the manufacturing process or enhance the overall appearance of the finished article. Examples of these compounds may be long chain fatty acids and their salts, organic and inorganic coloring agents, free radical inhibitors, pH buffering agents and other materials known to enhance processing of polymers within the polymer industry.

In another preferred embodiment of the present invention, solid materials may be incorporated into the polymer or resin mixtures. Such solid materials may be, for example, chopped carbon or glass fiber or nanotubes, carbon black, graphite powder, tale, mica, polyamide fiber and other fillers commonly used in the polymer industry. As is known in the polymer industry, such fillers may be advantageously added to a polymer matrix for the purposes of enhancing strength, durability, bulk density, machineablity of the resulting polymeric article. Of, course the above list is not exhaustive and other uses of the fillers may also be contemplated.

Devices

One preferred device produced of the polymeric product described herein may be a substitution for cartilage. The cartilage substitution may replace damaged cartilage between intact bones, or it may be part of a medical prosthesis comprising cartilage substitution.

A device produced of the polymeric product itself can be used as a growth medium and/or network for the natural or artificial cells, such as chondrocytes.

A device made from the polymeric product optionally comprising one or more metal components as described above Is capable of being formed to suit into parts of the organism as described elsewhere herein. Especially the device is suitable to be used in animals, such as mammals and human beings, preferred is human beings. The animals, to which the medical device may be utilised, may be selected from the group of mammals, such as but not limited to horses, dogs, cats, cows and monkeys.

In one embodiment the device is especially constructed to be utilised to support, hold, sustain, bear, carry, replace or displace any constitution within the mammalian body, which comprises high shape stability and good wear resistance.

The polymeric product optionally comprising one or more metal components is adapted not to interfere with intra-articular or other components when the device is in the body of a human.

The polymeric product as medical device may be but is not limited to be used as joint spacer implant in joints of knees, hip, shoulders, fingers, wrist elbow, spine, neck, loin, toes and ankles. Especially the devices may be used in diseased patients with osteoarthritic degeneration of joints. The implants with a smooth articulating surface oppose the diseased and degenerated cartilage joint facet, which is expected to lead to reduced force and stresses and improved mobility in the joint with consequent reduced pain and improved functional capacity of that joint.

The medical device as described herein may be produced in a number of sizes corresponding to the natural variety of the bones within the joint where it is intended to be used as well as to the differences in bone size due to the age or size of individuals.

Moreover, non-interference of the intra-articular components may be achieved by a hole which runs through the body of the device; that is to say the device may comprise a hole through which intra-articular components may pass. When loading the device, the slits may serve to pass intra-articular components through the body of the device. The slits in this embodiment run from the periphery of the body of the device to the hole through which the intra-articular components pass after the device is implanted or loaded.

Typically, and to at least some extent, the device is adapted in its structure and/or material composition to alleviate conditions associated with worn cartilage by providing a spacer function and/or to exert pressure distribution in the joint when the joint is loaded and/or to provide at least part of the sliding rotating movement of the joint by internal movement of at least part of the device.

It is also an object of the present invention to provide a method for non-invasive locking of a device within a joint. In addition, the method is independent of use of cement or bony ingrowth of the device.

The device may completely or substantially completely surround an intra-articular component or other components of the organism.

A device made from the polymeric product described above is capable of being formed to suit any joint cavity of animals or human beings, therefore the device may for example be formed to fit into any one of the following joints: Hip joint, knee joint, ankle joints, shoulder joint, elbow joints, wrist, fingers, spinal column joints, such as for substituting intervertebral discs, and the jaw joint.

The medical device may constitute the surface of a prosthetic device. It may be the entire surface or part of the surface of a prosthetic device Also the device may constitute a complete or part of a hip endo-prosthesis, or it may be a breast prosthesis, a stent, a catheter, a heart valve or cartilage substitution.

Generally, the invention comprises the polymeric product optionally comprising one or more metal components as described above from which different medical devices may be manufactured, also the method of producing the polymeric product and medical devices is enclosed within the invention. Enclosed are methods of producing a polymeric product and medical devices as described above, as well as any combination of the features described for the polymeric product and the medical devices.

In another aspect the invention relates to a method for producing a polymeric product optionally comprising one or more metal components, the method comprising obtaining a number of at least three polymer layers optionally comprising one or more metal components, and positioning the polymer layers in a sandwich composition, forming the sandwich composition of polymer layers by heating the composition followed by pressing it into a mould, where the heating and pressing processes are conducted in vacuum, and providing the polymeric product in a desired shape.

In the method for producing a polymeric product wherein the polymeric product is as described above at least three polymer layers may be utilised, these polymer layers may constitute a core with at least one layer of fabric on each side, where the core differs in constitution from the fabrics, preferred is the method for producing a polymeric product where the fabrics at the different sides of the core have equal constitutions.

The method for producing a polymeric product comprises two or more layers of fabrics, where the two or more layers of fabrics have a film of a polymer layer in between each fabric.

In the method for producing a polymeric product the core and the film have similar composition except for the thickness of the polymer layer. The thickness of the polymer layers is as described above, in a preferred embodiment the film is between 0.01 and 2 mm thick, and the core is between 0.1 and 10 mm thick.

In an embodiment the method for producing a polymeric product comprises fabric film and core where the structure of the fabric are composed of long polymer fibre, and the core and film are composed of short chain polymers. These polymer fibres can be selected among polyethylene (PE), polypropylene (PP) and polyvinylpyrrolidone (PVP). Most preferable is polyethylene (PE). The long polymer fibres are ultra high molecule weight polyethylene (UHMWPE) fibre and the short chain polymers may be branched.

In an embodiment the method for producing a polymeric product comprises fabric which is manufactured, e.g., woven, into a shape or form suitable for the shape of the polymeric product. The fabric consist of UHMWPE fibres in which the intersects are positioned as formerly described, preferably in angles of about 90 degree.

In an embodiment the method for producing a polymeric product comprises fabric which has high tensile strength and high wear resistance, and a core which absorbs shocks, pushes and strokes.

The method for producing a polymeric product comprises arranging the polymer layers in the order of fabric, film and core in accordance to the description above.

The most preferred constitutions are listed above. The polymer layers are heated, and under vacuum the polymeric product is pressed in to a mould. The device, which is formed, is treated by ionising radiation, to further cross bind the polymers and thereby improve the strength of the product. The product is flier subjected to annealing to ensure all linking has appeared.

In an embodiment the method for producing a polymeric product comprises surface coating of the annealed polymeric product and further the polymeric product is sterilised by ionising radiation or by heating.

In another embodiment the method for producing a polymeric product comprises annealing the polymeric product before it is subjected to surface coating.

In a further embodiment the method for producing a polymeric product comprises simultaneously cross-linking and sterilisation of the polymeric product by treating with ionising radiation or by heating.

In an embodiment the method for producing a polymeric product comprises surface coating of the polymeric product, as formerly described.

In a preferred embodiment the method for producing a polymeric product comprises production of the polymeric product where the shape and size of the polymeric product can be any possible to produce by pressing into to a mould, the mould forming a polymeric product which can be flat or round or in between and where the three-dimensional shape can be any possible forming by pressing into a mould.

The polymeric product can be utilised to produce a prosthetic device comprising polymer layers, the order of the polymer layers, and the method of production of the polymeric product as described above.

Preferred is a method of producing a prosthetic device of three polymer layers, which constitute a core with at least one layer of fabric on each side. Another preferred constitution is a core which at each side has two layers of fabric with a film in between. A further preferred constitution is a film between two layers of fabric.

In a preferable embodiment of the method the prosthetic device are produced from polymer layers optionally comprising one or more metal components composed of a polymer selected among polyethylene (PE), polypropylene (PP) and polyvinylpyrrolidone (PVP). Most preferable is a prosthetic device wherein the polymer layers are composed of polyethylene (PE).

In a further preferable embodiment of the method the prosthetic device are composed of fabrics of long polymer fibre, which preferable are ultra high molecule weight polyethylene (UHMWPE) fibre or other polyethylene fibre as previously described, whereas the core and the film are composed of short chain polymers, the short chain polymers may be branched.

The fabric Is of medical grade and is woven into a shape suitable for the shape of the polymeric product. The shaping and physical characteristics is determined by the arrangement of the UHMWPE fibres, the fibres can have intersects in angles as described formerly.

In a preferred embodiment the prosthetic device has a high tensile strength and a high wear resistance due to the properties of the fabrics, whereas the core absorbs shocks, pushes and strokes.

The polymeric constitution of the prosthetic device is obtained in accordance with the details given above where the polymer layers are heated, subjected to vacuum and pressed into shape in a mould, and further treated as described above.

### EXAMPLES

### Example 1

### Artificial Cartilage Cup

The artificial cartilage cup is an artificial joint spacer made to replace the missing or damaged cartilage so the joint can stay mobile.

The cup is based on a sandwich construction with a LDPE core reinforced on both sides with fibre fabric.

At the edge metal markers makes it possible to trace the cup when implanted.

The round LDPE collar of the cup makes a cup without sharp edges and captures the metal markers.

Finally a crosslinking of the polymer improves the performance of the LDPE core.

The production process may include the following steps:

Injection Moulding of Base LDPE Disk

The LDPE disk is made of pellets/granulates in the injection moulding process. The disk is approximately five mm thick and 134 min in diameter. One standard disk size will later be formed to different size of cups.

Pressure Consolidation with Fibre Fabric

Two pieces of 20×20 cm fibre fabric are placed on each side of the disk and the sandwich is pressed to form a cup with a surplus material like an irregular hat brim. Different cup sizes arm produced and identified by an individual number.

Shaping the Cup by Cutting Off Excess Material

Cutting off the hat brim leaves a cup with right angle edges.

Drilling of Cup Holes and Mounting of Metal Markers

Metal markers in the cup make tracing the cup in the body possible. For the first test production, the markers are tantalum balls, and for later production, the markers will be stainless steel balls.

Injection Moulding of LDPE-Collar on Cup

The metal markers are fixed, and LDPE-collar covers the right angle edges.

Packaging with Nitrogen Gas

In a packing machine, the cup is packed in an aluminum pouch with nitrogen gas, and the pouch is sealed to prevent oxygen from being in contact with the cup. Oxygen will hinder the later crosslinking process, as it reacts with the free radicals. The aluminum pouch is put in a shipment box, ready for sending to the crosslinking plant.

Crosslinking and Sterilization

The cup goes to the irradiation plant, is irradiated and returns to the production area. The irradiation forms free radicals. The free radicals are very reactive places in the polymer material, which react to form crosslinking in the polymer. The irradiation dose is about 200 kGy.

Annealing

Just after the irradiation process, there are still free radicals. In the annealing process, the free radicals form crosslinking. The annealing process is a heating at approximately 75° C., which speeds up the crosslinking reaction without softening the cup. The process is slowly running even at room temperature, but might take about one month. The temperature must be at a relatively low level in order to avoid softening and deforming of the cup.

Final Packing, Releasing and Storing

The cup is packed in inner box, labelled and instructions for use are supplied. The product is released after a quality check, and stored at the subcontractor.

1. A medical device comprising at least a first surface area, wherein at least a first polymeric component optionally further comprising one or more metal components is attached to said first surface area.

2. The device according to claim 1, wherein said at least first polymeric component comprises at least one fabric of one or more polymer fibre and/or of one or more metal fibre and/or a 3D network of polymer fibre and/or metal fibre.

3. The device according to claim 2, wherein said polymer fibre and/or said polymer optionally further comprising one or more metal components is a polyolefin, such as polyethylene, such as UHMWPE.

4. The device according to claim 1, wherein said metal fibre and/or said metal or metal alloy is selected from the group of titanium, gold, silver, chromium-cobaltum, zirconia, cobalt-chromium-molybdenum alloy and Stainless Steel alloys and/or a ceramic of one or more of these metals and alloys.

5. The device according to claim 1, wherein said device is an acetabular cup, a spacer to be located between an acetabular cup and a head of hip stem, or an interpositional arthroplasty.

6. The device according to claim 1, wherein said first surface area is a part or the entire surface area of a first volume, where said first volume is smaller than the final volume and where said final volume is the volume of the device before implantation, and wherein the difference between said final volume and said first volume is a volume made up by at least said first polymeric component optionally further comprising one or more metal components.

7. The device according to claim 6, wherein said first volume has a shape which corresponds to the final volume of the device before implantation.

8. The device according to claim 6, wherein said first volume corresponds to at least 50% of said final volume, such as at least 60%, such as at least 70%, such as at least 80%, such as at least 85%, such as at least 90%, such as at least 95%, such as at least 97%, such as at least 2%.

9. The device according to claim 6, wherein said first volume corresponds to a lesser degree to said final volume in areas where said at least first polymeric component optionally further comprising one or more metal components is attached.

10. The device according to claim 1, wherein said device at least in said first area is subjected to wear when said device is located in an individual.

11. The device according to claim 6, wherein said first volume of said device is made partly or entirely of polymer, bone and/or metal.

12. The device according to claim 1, wherein said first polymeric component optionally further comprising one or more metal components constitute at least 1% of the entire outer surface of the device, such as at least 3%, such as at least 5%, such as at least 8%, such as at least 10%, such as at least 15%, such as at least 20%, such as at least 25%, such as at least 30%, such as at least 35%, such as at least 40%, such as at least 50%, such as at least 60%, such as at least 70%, such as at least 80%, such as at least 90%, such as at least 2%.

13. The device according to claim 2, wherein said fabric is made by weaving, knitting and/or crocheting.

14. The device according to claim 1, wherein said device further comprises at least a second polymeric component optionally further comprising one or more metal components which is attached at least to said first surface area, and wherein the chain length of the first polymeric component is longer than the chain length of the second polymeric component.

15. The device according to claim 1, comprising at least a first unit with at least a convex surface and a second unit with at least a concave surface, where said convex and concave surfaces are congruent with each other and said first unit fits partially or entirely into said second unit, and wherein said first and/or second unit comprises at least one polymeric component optionally further comprising one or more metal components.

16. The device according to claim 15, wherein said first and second units have substantially similar size.

17. The device according to claim 15, wherein said first and second units have different sizes.

18. The device according to claim 15, wherein said first and second units are connected or substantially connected by said convex and concave surface.

19. The device according to claim 18, wherein said concave surface is at least 0.01 mm smaller than said convex surface.

20. The device according to claim 15, wherein said units are movable compared to each other, and said units continue to be in contact during a movement and/or return to be in contact when a movement is finished.

21. The device according to claim 15, wherein said first unit and/or said second unit is attached to an implant and/or a bone.

22. The device according to claim 21, wherein said device is part of a hip joint prosthesis, and where said device comprises a spacer e.g. a cup shaped spacer which is positioned between the natural femoral stem or a metal femoral stem and a natural acetabular cup or a prosthetic acetabular cup.

23. The device according to claim 1, wherein said one or more metal components are in the a powder, granulate, chopped fibres, long fibres, 2D structural components like plates, 3D structural components like shaped plates or hemicircles with holes.
